(19) United States
(12) Patent Application Publication
(10) Pub. No.: US 2010/0166940 A1
(43) Pub. Date: Jul. 1, 2010

(54) USE OF LOW PH TO MODIFY THE TEXTURE OF STRUCTURED PLANT PROTEIN PRODUCTS

(75) Inventors: Matthew K. McMindes, Chesterfield, MI (US); Valdomiro Valle, Jandra-Sao Paulo (BR); Mac W. Orcutt, St. Louis, MO (US); Kurt A. Busse, Florissant, MO (US)

Correspondence Address: Solae, LLC, 4300 Duncan Avenue, Legal Department E4, St. Louis, MO, 63110, US

(73) Assignee: Solae LLC

(21) Appl. No.: 12/444,111

(22) PCT Filed: Oct. 5, 2007
(86) PCT No.: PCT/US07/80601

§ 371 Date: Apr. 2, 2009

Related U.S. Application Data
(60) Provisional application No. 60/828,298, filed on Oct. 5, 2006.

(30) Foreign Application Priority Data
Oct. 5, 2007 (US) 11868087

(51) Int. Cl. A23J 3/14; C07K 1/107; A23L 1/31

(52) U.S. Cl. 426/641; 530/402; 426/516; 426/656

(57) ABSTRACT

The invention provides animal meat compositions and simulated animal meat compositions. In addition, the invention provides a process for producing animal meat compositions and simulated animal meat compositions. The process comprises producing the animal meat compositions and simulated animal meat compositions under conditions of low pH.

### CROSS REFERENCE TO RELATED APPLICATIONS

This application claims priority from Provisional Application Ser. No. 60/828,298 filed on Oct. 5, 2006 and PCT International Application No. PCT/US2007/080601 filed on Oct. 5, 2007, which are hereby incorporated by reference in their entirety.

### FIELD OF THE INVENTION

The present invention provides animal meat compositions and simulated meat compositions. The invention also provides processes for producing the animal meat compositions and simulated animal meat compositions. In the process, a pH-lowering agent is generally utilized.

### BACKGROUND OF THE INVENTION

Food scientists have devoted much time developing methods for preparing acceptable meat-like food products, such as beef, pork, poultry, fish, and shellfish analogs, from a wide variety of plant proteins. Soy protein has been utilized as a protein source because of its relative abundance and reasonably low cost. Extrusion processes typically prepare meat analogs. The dry blend is processed to form a fibrous material. To date, meat analogs made from high protein extrudates have had limited acceptance because they lack meat-like texture characteristics and mouth feel. Rather, they are characterized as spongy and chewy, largely due to the random, twisted nature of the protein fibers that are formed. Most are used as extenders for ground, hamburger-type meats.

There is a still an unmet need for a structured plant protein product that simulates the fibrous structure of animal meat and has an acceptable meat-like texture, flavor and color.

### SUMMARY OF THE INVENTION

One aspect of the invention provides a process for producing a structured plant protein product. The process typically comprises combining a plant approximately 6.0. The mixture is extruded under conditions of elevated temperature and pressure to form a structured plant protein product comprising protein fibers that are substantially aligned.

Another aspect is a process for producing an animal meat composition. The process typically comprises combining animal meat, plant protein material with a pH-lowering agent to form a mixture having a pH below approximately 6.0. The mixture is then extruded under conditions of elevated temperature and pressure to form a structured plant protein product comprising protein fibers that are substantially aligned.

Yet another aspect of the invention provides an animal meat composition. In general, the animal meat composition comprises animal meat, a pH-lowering agent, and a structured plant protein product comprising protein fibers that are substantially aligned.

A further aspect of the invention provides a simulated animal meat composition. The simulated animal meat composition comprises a structured plant protein product comprising protein fibers that are substantially aligned and a pH-lowering agent.

### REFERENCE TO COLOR FIGURES

The application contains at least one photograph executed in color. Copies of this patent application publication with color photographs will be provided by the Office upon request and payment of the necessary fee.

### FIGURE LEGENDS

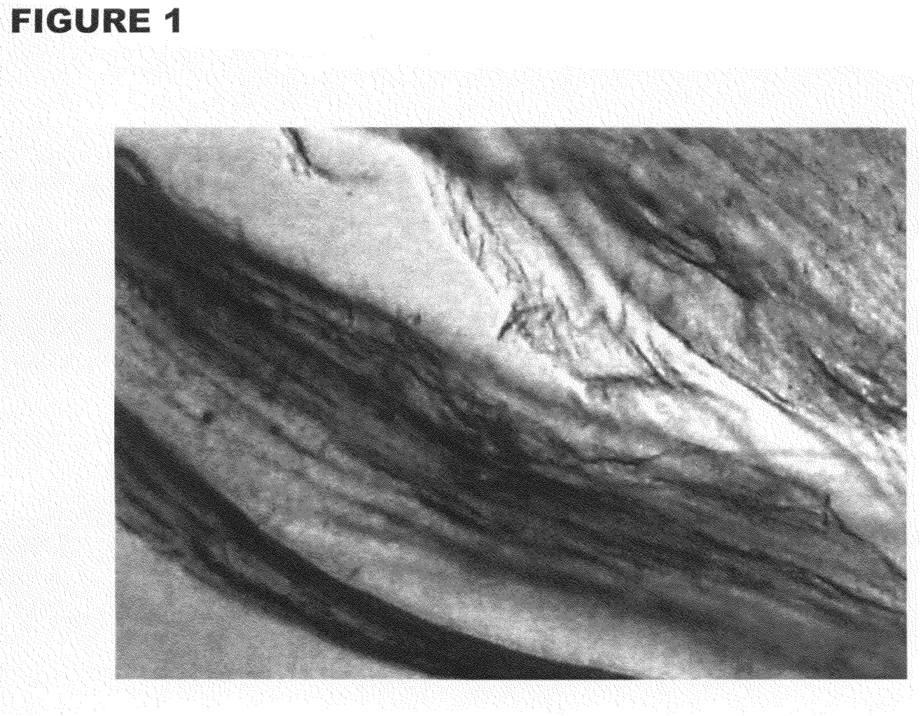

FIG. 1—depicts a photographic image of a micrograph showing a structured plant protein product of the invention having protein fibers that are substantially aligned.

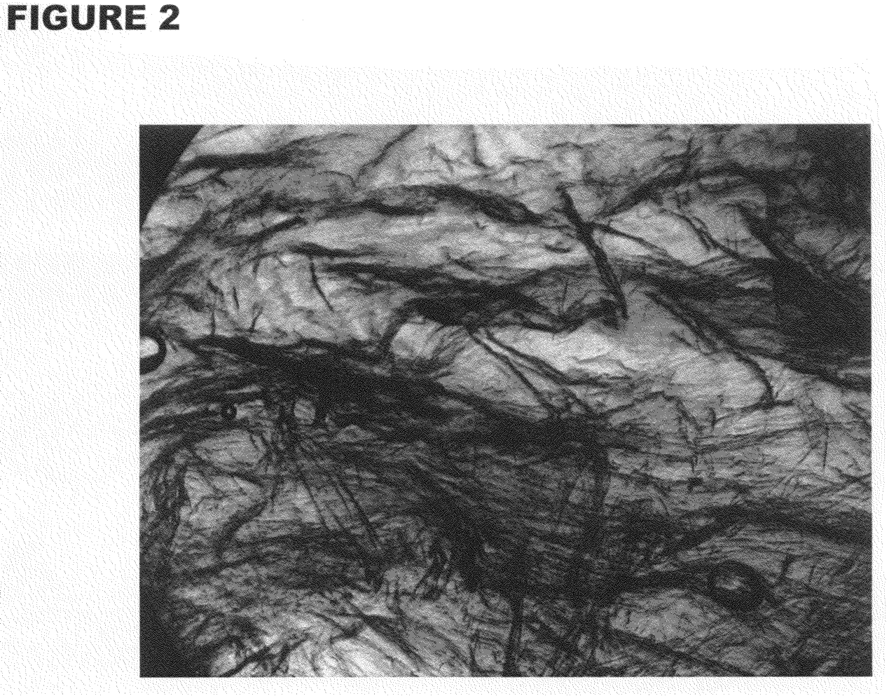

FIG. 2—depicts a photographic image of a micrograph showing a plant protein product not produced by the process of the present invention. The protein fibers comprising the plant protein product, as described herein, are crosshatched.

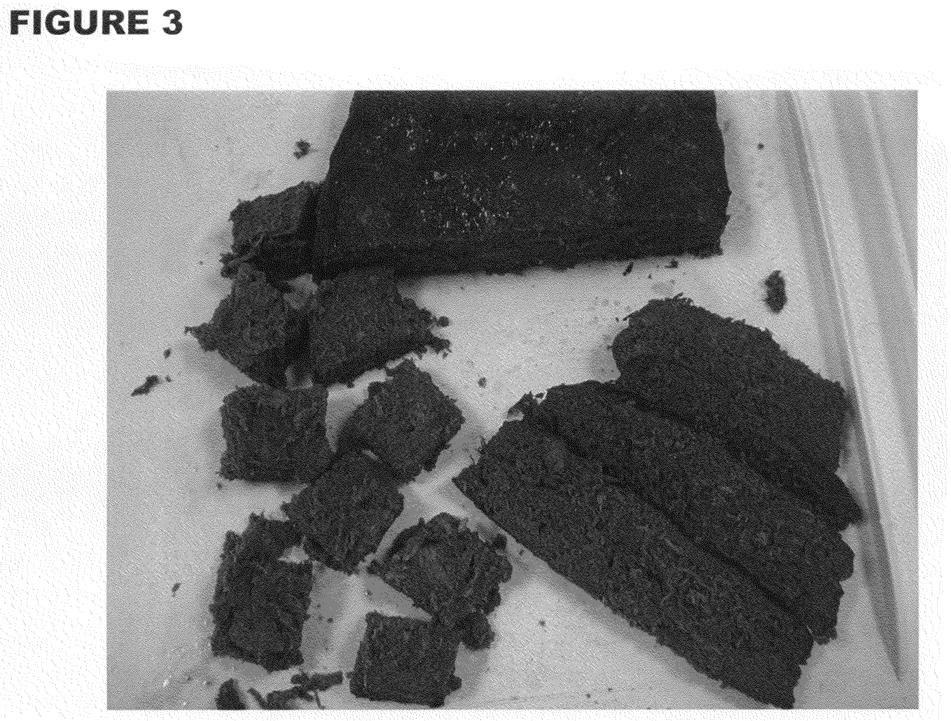

FIG. 3—depicts a photographic image of an animal meat composition in which the pH of the composition was reduced to 5.6 with lactic acid during its manufacture.

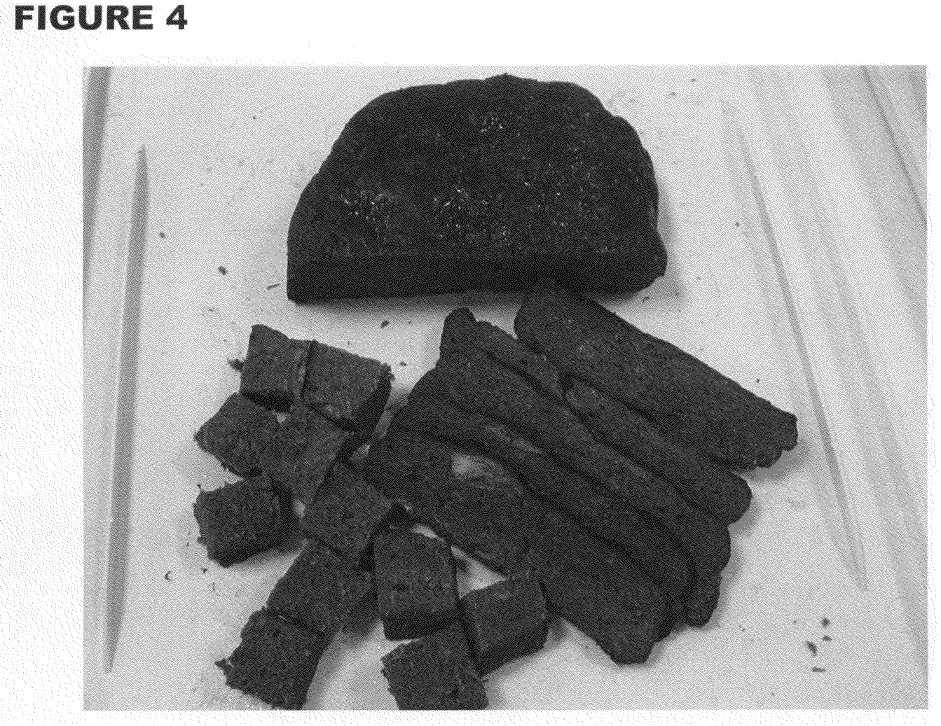

FIG. 4—depicts a photographic image of an animal meat composition in which the pH of the composition was reduced to 6.7 during its manufacture.

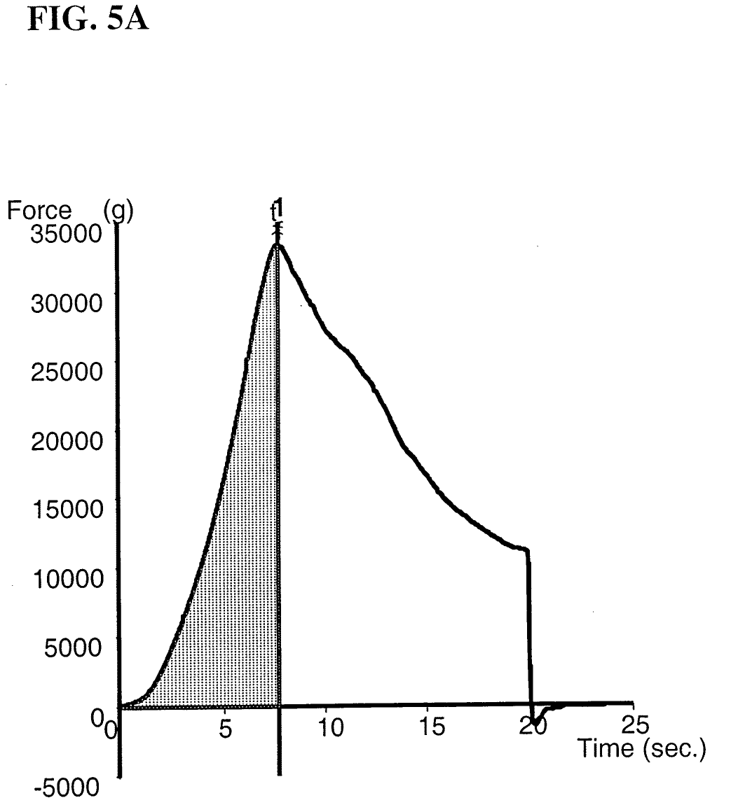

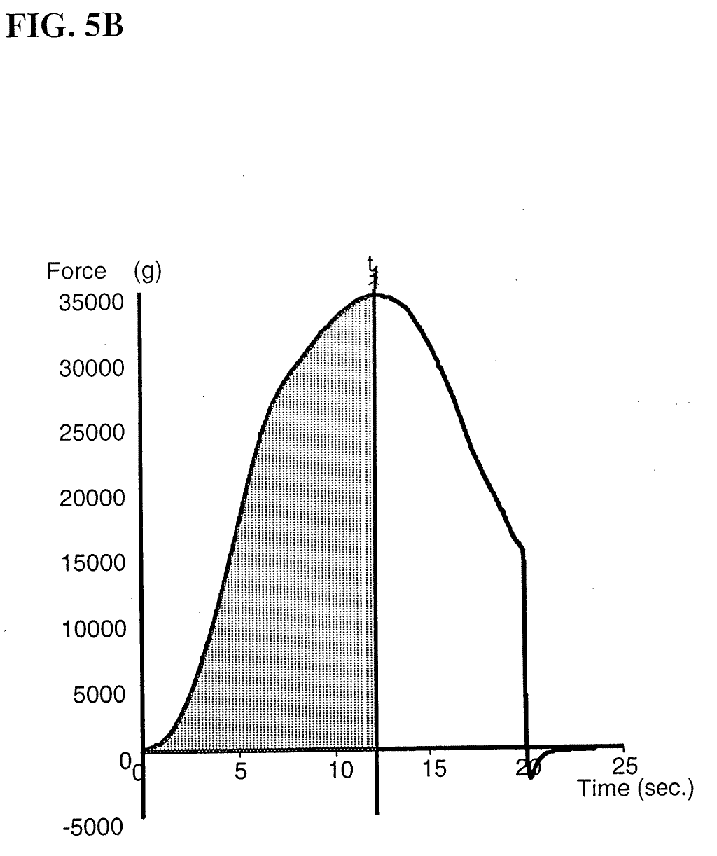

FIGS. 5a and 5b—are graphs demonstrating the relationship between time and force for the shear force test, with 5a representing a sample that does not include the pH lowering agent and 5b representing a sample that includes the pH lowering agent.

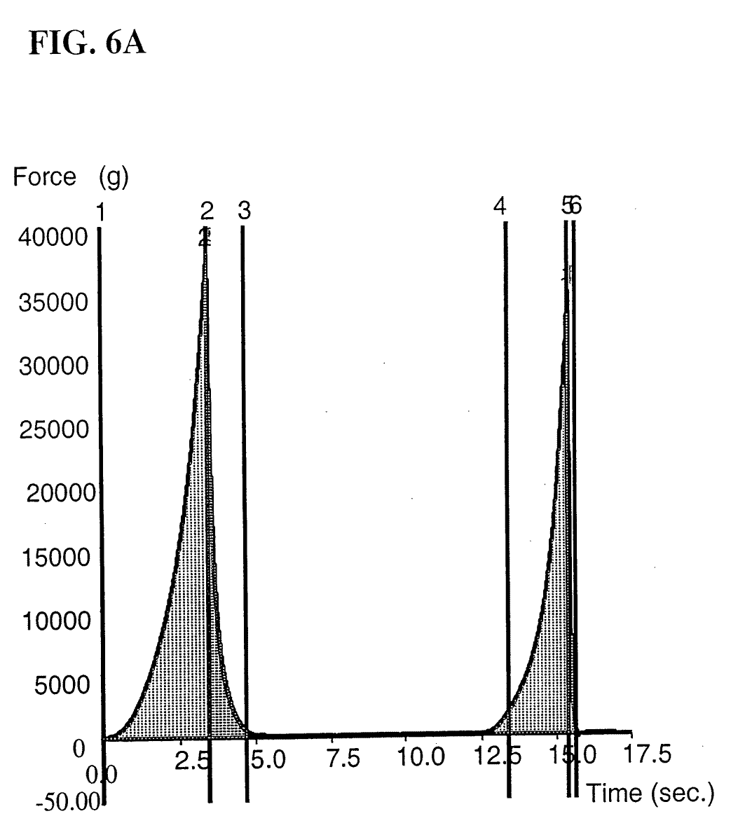

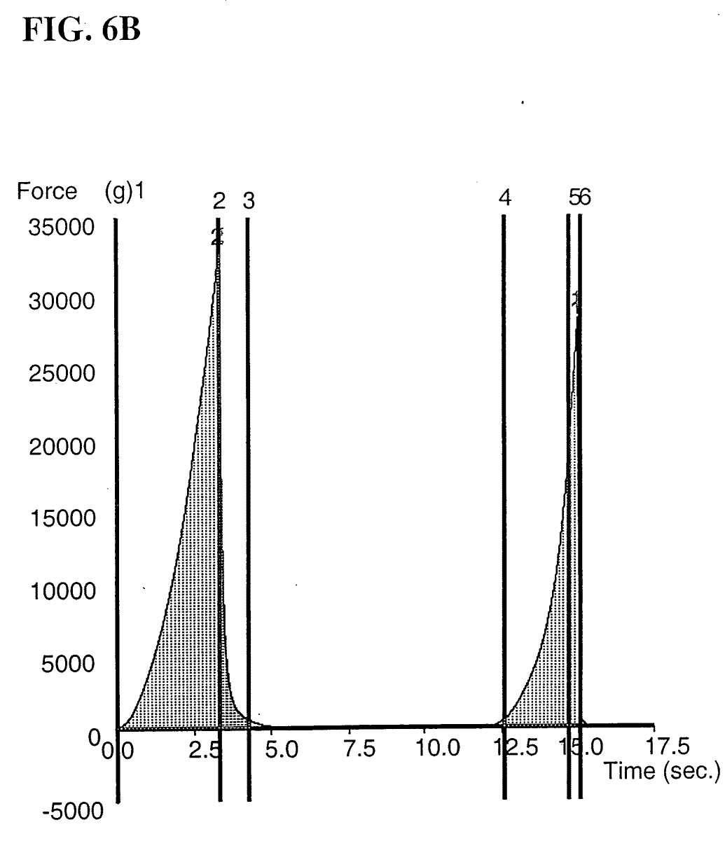

FIGS. 6a and 6b—are graphs demonstrating the texture profile analysis for a sample that does not include the pH lowering agent (6a) and a sample that includes the pH lowering agent (6b).

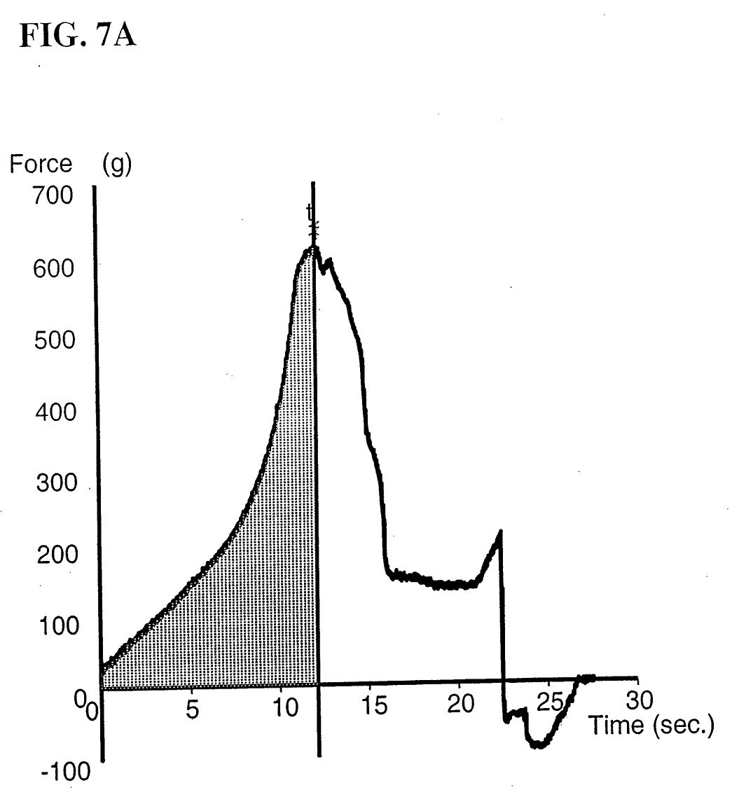

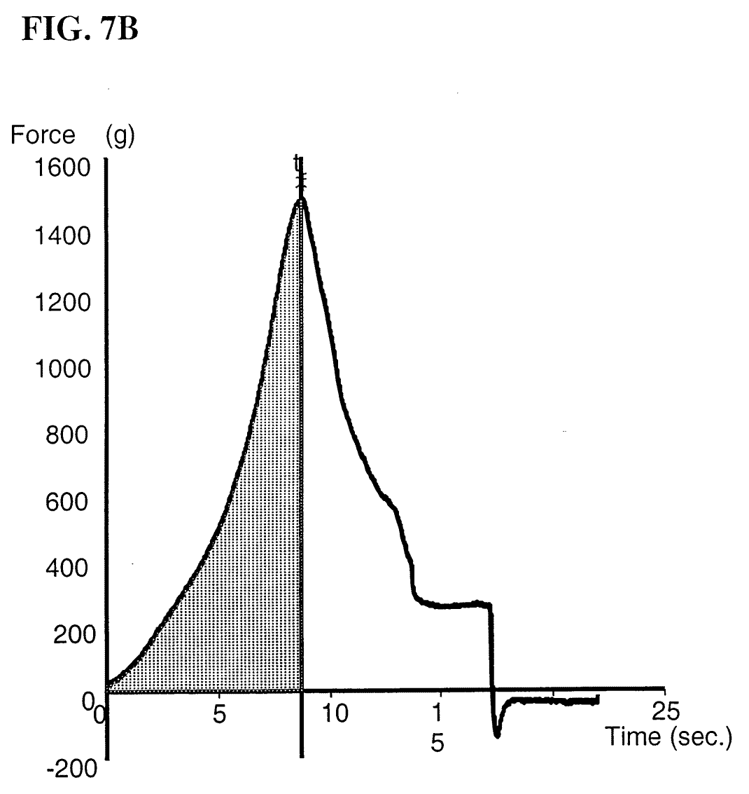

FIG. 7a—is a graph, demonstrating the shear force test for a sample with a pre-retort pH of 6.74.

FIG. 7b—is a graph demonstrating the shear force test for a sample with a pre-retort pH of 5.46.

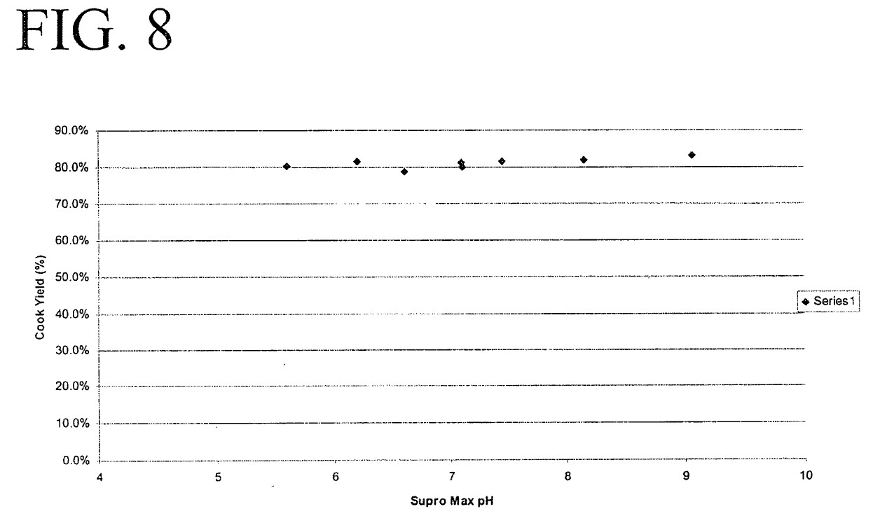

FIG. 8—is a graph demonstrating percentage of yield after cooking for meat blends with varying pH levels.

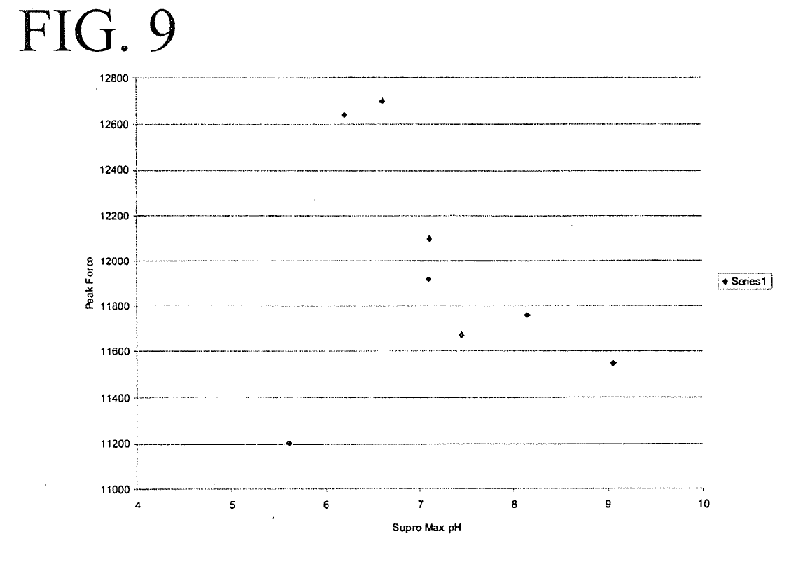

FIG. 9—is a graph demonstrating shear force (peak force) for meat blends with varying pH levels.

### DETAILED DESCRIPTION OF THE INVENTION

The present invention provides animal meat compositions or simulated meat compositions. Typically, both compositions include structured plant protein products comprising protein fibers that are substantially aligned. The compositions may optionally include animal meat. The invention also provides a process for producing the compositions under conditions of acidic pH. It has been discovered that producing an animal meat composition or a simulated animal meat composition under conditions of low pH, such as at the pH level found in rigor meat, results in meat composition with improved meat-like qualities. By way of example referring to FIGS. 3 and 4, the animal meat composition depicted in FIG. 3 was prepared at an acidic pH of 5.6, while the animal meat composition of FIG. 4 was prepared at a relatively neutral pH of 6.7. As shown in the photographic images, the animal meat composition produced under acidic conditions has a consistency that is fibrous, and has a more meat-like texture compared to the animal meat composition produced under neutral pH conditions, which has a more gummy and less cohesive consistency. Because of the improved texture and flavor provided by the pH reduction, the compositions of the invention may be utilized in a variety of applications to simulate whole muscle meat.

### (I) Structured Plant Protein Products

The animal meat compositions and simulated animal meat compositions of the invention each comprise structured plant protein products comprising protein fibers that are substantially aligned, as described in more detail in I(f) below. In an exemplary embodiment, the structured plant protein products are extrudates of plant materials that have been subjected to the extrusion process detailed in I(e) below. Because the structured plant protein products utilized in the invention have protein fibers that are substantially aligned in a manner similar to animal meat, the animal meat compositions and simulated animal meat compositions generally have the texture and feel of compositions containing all animal meat producing the meat-like texture consumers seek.

(a) Protein-Containing Starting Materials

A variety of ingredients that contain protein may be utilized in an extrusion process to produce structured plant protein products suitable for use in animal meat compositions and simulated animal meat compositions. While ingredients comprising proteins derived from plants are typically used, it is also envisioned that proteins derived from other sources, such as animal sources, may be utilized without departing from the scope of the invention. For example, a dairy protein selected from the group consisting of casein, caseinates, whey protein, milk protein concentrate, milk protein isolate, and mixtures thereof may be utilized. In an exemplary embodiment, the dairy protein is whey protein. By way of further example an egg protein selected from the group consisting of ovalbumin, ovoglobulin, ovomucin, ovomucoid, ovotransferrin, ovovitella, ovovitellin, albumin globulin, and vitellin may be utilized.

It is envisioned that other ingredient types in addition to proteins may be utilized. Not limiting examples of such ingredients include sugars, starches, oligosaccharides, soy fiber and other dietary fibers, and gluten.

It is also envisioned that the protein-containing starting materials may be gluten-free. Because gluten is typically used in filament formation during the extrusion process, if a gluten-free starting material is used, an edible crosslink agent may be utilized to facilitate filament formation. Non-limiting examples of suitable crosslink agents include Konjac glucomannan (KGM) flour, edible crosslink agents such as transglutanimnase, beta glucan, such as Pureglucan® manufactured by Takeda (USA), calcium salts, and magnesium salts. One skilled in the art can readily determine the amount of cross linker needed, if any, in gluten-free embodiments.

Irrespective of its source or ingredient classification, the ingredients utilized in the extrusion process are typically capable of forming extrudates (structured plant protein products) having protein fibers that are substantially aligned. Suitable examples of such ingredients are detailed more fully below.

(i) Plant Protein Materials

In an exemplary embodiment, at least one ingredient derived from a plant will be utilized to form the protein-containing materials. Generally speaking, the ingredient will comprise a protein. The amount of protein present in the ingredient(s) utilized can and will vary depending upon the application. For example, the amount of protein present in the ingredient(s) utilized may range from about 40% to about 100% by weight. In another embodiment, the amount of protein present in the ingredient(s) utilized may range from about 50% to about 100% by weight. In an additional embodiment, the amount of protein present in the ingredient(s) utilized may range from about 60% to about 100% by weight. In a further embodiment, the amount of protein present in the ingredient(s) utilized may range from about 70% to about 100% by weight. In still another embodiment, the amount of protein present in the ingredient(s) utilized may range from about 80% to about 100% by weight. In a further embodiment, the amount of protein present in the ingredient(s) utilized may range from about 90% to about 100% by weight.

The ingredient(s) utilized in extrusion may be derived from a variety of suitable plants. By way of non-limiting example, suitable plants include legumes, corn, peas, canola, sunflowers, sorghum, rice, amaranth, potato, tapioca, arrowroot, canna, lupin, rapeseed, wheat, oats, rye, barley, and mixtures thereof.

In one embodiment, the ingredients are isolated from wheat and soybeans. In another exemplary embodiment, the ingredients are isolated from soybeans. Suitable wheat derived protein-containing ingredients include wheat gluten, wheat flour, and mixtures thereof. An example of commercially available wheat gluten that may be utilized in the invention is Gem of the West Vital Wheat Gluten, either regular or organic, each of which is available from Manildra Milling (Shawnee Mission, Kans.). Suitable soybean derived protein-containing ingredients (“soy protein material”) include soy protein isolate, soy protein concentrate, soy flour, and mixtures thereof, each of which are detailed below. In each of the foregoing embodiments, the soybean material may be combined with one or more ingredients selected from the group consisting of a starch, flour, gluten, a dietary fiber, and mixtures thereof.

Suitable examples of protein-containing material isolated from a variety of sources are detailed in Table A, which, shows various protein ingredient combinations.

TABLE A
Protein Combinations

| First protein source | Second ingredient |
|---|---|
| soybean | wheat |
| soybean | dairy |
| soybean | egg |
| soybean | corn |
| soybean | rice |
| soybean | barley |
| soybean | sorghum |
| soybean | oat |
| soybean | millet |
| soybean | rye |
| soybean | triticale |
| soybean | buckwheat |
| soybean | pea |
| soybean | peanut |
| soybean | lentil |
| soybean | lupin |
| soybean | channa (garbonzo) |
| soybean | rapeseed (canola) |
| soybean | cassava |
| soybean | sunflower |
| soybean | whey |
| soybean | tapioca |
| soybean | arrowroot |
| soybean | amaranth |
| soybean | wheat and dairy |
| soybean | wheat and egg |
| soybean | wheat and corn |
| soybean | wheat and rice |
| soybean | wheat and barley |
| soybean | wheat and sorghum |
| soybean | wheat and oat |
| soybean | wheat and millet |
| soybean | wheat and rye |
| soybean | wheat and triticale |
| soybean | wheat and buckwheat |
| soybean | wheat and pea |
| soybean | wheat and peanut |
| soybean | wheat and lentil |
| soybean | wheat and lupin |
| soybean | wheat and channa (garbonzo) |
| soybean | wheat and rapeseed (canola) |
| soybean | wheat and cassava |
| soybean | wheat and sunflower |
| soybean | wheat and potato |
| soybean | wheat and tapioca |
| soybean | wheat and arrowroot |
| soybean | wheat and amaranth |
| soybean | corn and wheat |
| soybean | corn and dairy |
| soybean | corn and egg |
| soybean | corn and rice |
| soybean | corn and barley |
| soybean | corn and sorghum |
| soybean | corn and oat |
| soybean | corn and millet |
| soybean | corn and rye |
| soybean | corn and triticale |
| soybean | corn and buckwheat |
| soybean | corn and pea |
| soybean | corn and peanut |
| soybean | corn and lentil |
| soybean | corn and lupin |
| soybean | corn and channa (garbonzo) |
| soybean | corn and rapeseed (canola) |
| soybean | corn and cassava |
| soybean | corn and sunflower |
| soybean | corn and potato |
| soybean | corn and tapioca |
| soybean | corn and arrowroot |
| soybean | corn and amaranth |

In each of the embodiments delineated in Table A, the combination of protein-containing materials may be combined with one or more ingredients selected from the group consisting of a starch, flour, gluten, a dietary fiber, and mixtures thereof. In one embodiment, the protein-containing material comprises protein, starch, gluten, and fiber. In an exemplary embodiment, the protein-containing material comprises from about 45% to about 65% soy protein on a dry matter basis; from about 20% to about 30% wheat gluten on a dry matter basis; from about 10% to about 15% wheat starch on a dry matter basis; and from about 1% to about 5% fiber on a dry matter basis. In each of the foregoing embodiments, the protein-containing material may comprise dicalcium phosphate, L-cysteine or combinations of both dicalcium phosphate and L-cysteine.

(ii) Soy Protein Materials

In an exemplary embodiment, as detailed above, soy protein isolate, soy protein concentrate, soy flour, and mixtures thereof may be utilized in the extrusion process. The soy protein materials may be derived from whole soybeans in accordance with methods generally known in the art. The whole soybean may be standard soybeans (i.e., non genetically modified soybeans), commoditized soybeans, hybridized soybeans, genetically modified soybeans, preserved soybeans, and combinations thereof.

Generally speaking, when soy isolate is used, an isolate is preferably selected that is not a highly hydrolyzed soy protein isolate. In certain embodiments, highly hydrolyzed soy protein isolates, however, may be used in combination with other soy protein isolates provided that the highly hydrolyzed soy protein isolate content of the combined soy protein isolates is generally less than about 40% of the combined soy protein isolates, by weight. In another embodiment, a membrane filtered soy isolate may be used. Examples of soy protein isolates that are useful in the present invention are commercially available, for example, from Solae, LLC (St. Louis, Mo.), and include SUPRO® 500E, SUPRO® EX 33, SUPRO® 620, and SUPRO® 545. In an exemplary embodiment, a form of SUPRO® 620 is utilized as detailed in Example 3.

Alternatively, soy protein concentrate or soy flour may be blended with the soy protein isolate to substitute for a portion of the soy protein isolate as a source of soy protein material. Typically, if a soy protein concentrate is substituted for a portion of the soy protein isolate, the soy protein concentrate is substituted for up to about 55% of the soy protein isolate by weight. In another embodiment, the soy protein concentrate is substituted for up to 50% of the soy protein isolate by weight. In another embodiment, the substitute is 40% by weight of the soy protein. In another embodiment, the amount substituted is up to about 30% of the soy protein isolate by weight. Examples of suitable soy protein concentrates useful in the invention include Procon, Alpha 12 and Alpha 5800, which are commercially available from Solae, LLC (St. Louis, Mo.). If a soy flour is substituted for a portion of the soy protein isolate, the soy flour is substituted for up to about 35% of the soy protein isolate by weight. The soy flour should be a high protein dispersibility index (PDI) soy flour.

Any fiber known in the art that will work in the application can be used as the fiber source. Soy cotyledon fiber may optionally be utilized as a fiber source. Typically, suitable soy cotyledon fiber will generally effectively bind water when the mixture of soy protein and soy cotyledon fiber is extruded. In this context, “effectively bind water” generally means that the soy cotyledon fiber has a water holding capacity of at least 5.0 to about 8.0 grams of water per gram of soy cotyledon fiber, and preferably the soy cotyledon fiber has a water holding capacity of at least about 6.0 to about 8.0 grams of water per gram of soy cotyledon fiber. When present in the soy protein material, soy cotyledon fiber may be present in an amount ranging from about 1% to about 20%, preferably from about 1.5% to about 20% and most preferably, from about 2% to about 5% by weight on a moisture free basis. Suitable soy cotyledon fiber is commercially available. For example, FIBRIM® 1260 and FIBRIM® 2000 are soy cotyledon fiber materials that are commercially available from Solae, LLC (St. Louis, Mo.).

(b) pH-Reducing Agent

The animal meat compositions and simulated meat compositions are generally produced under conditions of low pH, such as at the pH of, post rigor meat. In general, a low pH is achieved by contacting the composition with a pH-lowering agent. It is envisioned that the pH-lowering agent may be suitably contacted with the compositions, or products forming the composition, at various stages of the composition's manufacture. In one embodiment, the pH-lowering agent is contacted with the plant protein material and the mixture is then extruded according to the process detailed in I(e). Alternatively, the pH-lowering agent may be contacted with the structured plant protein product after it has been extruded, as detailed below in II and III.

Irrespective of the stage of manufacture at which the pH-lowering agent is introduced, suitable agents include those that will lower the pH of the composition to approximately the pH level of post rigor meat. As will be appreciated by a skilled artisan, the pH of post rigor meat can and will vary from animal to animal, but the pH will generally be acidic (i.e., below approximately 7.0). In one embodiment, the pH is below approximately 7.0. In another embodiment, the pH is between about 6.0 to about 7.0. In still another embodiment, the pH is below approximately 6.0. In another embodiment, the pH is between about 5.0 and about 6.0. In one alternative of this embodiment, the pH is between about 5.2 to about 5.9. In still another alternative of this embodiment, the pH is between about 5.4 to about 5.8. In an additional alternative of this embodiment, the pH is about 5.6. In another embodiment, the pH is below approximately 5.0. In a further embodiment, the pH is between about 4.0 to about 5.0. In still another embodiment, the pH is below approximately 4.0.

Several pH-lowering agents are suitable for use in the invention. The pH-lowering agent may be organic. Alternatively, the pH-lowering agent may be inorganic. In exemplary embodiments, the pH-lowering agent is a food grade edible acid. Non-limiting acids suitable for use in the invention include acetic, lactic, hydrochloric, phosphoric, citric, tartaric, malic, and combinations thereof.

As will be appreciated by a skilled artisan, the amount of pH-lowering agent utilized in the process of the invention can and will vary depending upon several parameters, including, the agent selected, the desired pH, and the stage of manufacture at which the agent is added. By way of non-limiting example, the amount of pH-lowering agent combined with the plant protein material (i.e., for application where the agent is added before extrusion of the mixture), or either of the animal meat composition or simulated meat composition (i.e., for applications where the agent is added after extrusion) may range from about 0.1% to about 15% on a dry matter basis. In another embodiment, the amount of pH-lowering agent may range from about 0.5% to about 10% on a dry matter basis. In an additional embodiment, the amount of pH-lowering agent may range from about 1% to about 5% on a dry matter basis. In other embodiments, the amount of pH-lowering agent may range from about 2% to about 3% on a dry matter basis. In another embodiment, the amount of pH-lowering agent is about 2.5% on a dry matter basis.

### (c) Additional Ingredients

A variety of additional ingredients may be added to any of the combinations of protein-containing materials and pH lowering agents above without departing from the scope of the invention. For example, antioxidants, antimicrobial agents, and combinations thereof may be included. Antioxidant additives include BHA, BHT, TBHQ, vitamins A, C and E and derivatives, and various plant extracts such as those containing carotenoids, tocopherols or flavonoids having antioxidant properties, may be included to increase the shelf-life or nutritionally enhance the animal meat compositions or simulated meat compositions. The antioxidants and the antimicrobial agents may have a combined presence at levels of from about 0.01% to about 10%, preferably, from about 0.05% to about 5%, and more preferably from about 0.1% to about 2%, by weight of the protein-containing materials that will be extruded.

### (d) Moisture Content

As will be appreciated by the skilled artisan, the moisture content of the protein-containing materials can and will vary depending upon the thermal process the combination is subjected to, e.g., retort cooking, microwave cooking, and extrusion.

In an exemplary embodiment, the thermal process is extrustion. Generally speaking, when the thermal process is extrusion, the moisture content may range from about 1% to about 80% by weight. In low moisture extrusion applications, the moisture content of the protein-containing materials may range from about 1% to about 35% by weight. Alternatively, in high moisture extrusion applications, the moisture content of the protein-containing materials may range from about 35% to about 80% by weight. In an exemplary embodiment, the extrusion application utilized to form the extrudates is low moisture. An exemplary example of a low moisture extrusion process to produce extrudates having proteins with, fibers that are substantially aligned is detailed in V) and Example 3 and 4.

### (e) Extrusion of the Protein-Containing Plant Material

A suitable extrusion process for the preparation of plant protein material comprises introducing the plant protein material and other ingredients into a mixing tank (i.e., an ingredient blender) to combine the ingredients and form a dry blended plant protein material pre-mix. As detailed above, in certain embodiments the pH-lowering agent may be contacted with the plant material before the mixture is extruded. The dry blended plant protein material pre-mix is then transferred to a hopper from which the dry blended ingredients are introduced along with moisture into a pre-conditioner to form a conditioned plant protein material mixture. The conditioned material is then fed to an extruder in which the plant protein material mixture is heated under mechanical pressure generated by the screws of the extruder to form a molten extrusion mass. The molten extrusion mass exits the extruder through an extrusion die.

(i) Extrusion Process Conditions

Among the suitable extrusion apparatuses useful in the practice of the present invention is a double barrel, twin-screw extruder as described, for example, in U.S. Pat. No. 4,600,311. Further examples of suitable commercially available extrusion apparatuses include a CLEXTRAL Model BC-72 extruder manufactured by Clextral, Inc. (Tampa, Fla.); a WENGER Model TX-57 extruder, a WENGER Model TX-168 extruder, and a WENGER Model TX-52 extruder all manufactured by Wenger Manufacturing, Inc. (Sabetha, Kans.). Other conventional extruders suitable for use in this invention are described, for example, in U.S. Pat. Nos. 4,763,569, 4,118,164, and 3,117,006, which are hereby incorporated by reference in their entirety. A single-screw extruder could also be used in the present invention. Examples of suitable, commercially available single-screw extrusion apparatuses include the Wenger X-175, the Wenger X-165, and the Wenger X-85 all of which are available from Wenger Manufacturing, Inc.

The screws of a twin-screw extruder can rotate within the barrel in the same or opposite directions. Rotation of the screws in the same direction is referred to as single flow or co-rotating whereas rotation of the screws in opposite directions is referred to as double flow or counter-rotating. The speed of the screw or screws of the extruder may vary depending on the particular apparatus; however, it is typically from about 250 to about 450 revolutions per minute (rpm). Generally, as the screw speed increases, the density of the extrudate will decrease. The extrusion apparatus contains screws assembled from shafts and worm segments, as well as mixing lobe and ring-type shearing elements as recommended by the extrusion apparatus manufacturer for extruding plant protein material.

The extrusion apparatus generally comprises a plurality of heating zones through which the protein mixture is conveyed under mechanical pressure prior to exiting the extrusion apparatus through an extrusion die. The temperature in each successive heating zone generally exceeds the temperature of the previous heating zone by between about 10° C. to about 70° C. In one embodiment, the conditioned pre-mix is transferred through four heating zones within the extrusion apparatus, with the protein mixture heated to a temperature of from about 100° C. to about 150° C. such that the molten extrusion mass enters the extrusion die at a temperature of from about 100° C. to about 150° C. One skilled in the art could adjust the temperature either heating or cooling to achieve the desired properties. Typically, temperature changes are due to work input and can happen suddenly.

The pressure within the extruder barrel is, typically between about 50 psig to about 500 psig, preferably between about 75 psig to about 200 psig. Generally, the pressure within the last two heating zones is from about 100 psig to about 3000 psig, preferably between about 150 psig to about 500 psig. The barrel pressure is dependent on numerous factors including, for example, the extruder screw speed, feed rate of the mixture to the barrel, feed rate of water to the barrel, and the viscosity of the molten mass within the barrel.

Water is injected into the extruder barrel to hydrate the plant protein material mixture and promote texturization of the proteins. As an aid in forming the molten extrusion mass, the water may act as a plasticizing agent. Water may be introduced to the extruder barrel via one or more injection jets. Typically, the mixture in the barrel contains from about 15% to about 35% by weight water. The rate of introduction of water to the barrel is generally controlled to promote production of an extrudate having desired characteristics. It has been observed that as the rate of introduction of water to the barrel decreases, the density of the extrudate decreases. Typically, less than about 1 kg of water per kg of protein is introduced to the barrel. Preferably, from about 0.1 kg to about 1 kg of water per kg of protein are introduced to the barrel.

(ii) Preconditioning

In a pre-conditioner, the protein-containing material and other ingredients can be preheated, contacted with moisture, and held under controlled temperature and pressure conditions to allow the moisture to penetrate and soften the individual particles. In another embodiment, in the pre-conditioner the pressure condition is ambient. The preconditioner contains one or more paddles to promote uniform mixing of the protein and transfer of the protein mixture through the preconditioner. The configuration and rotational speed of the paddles vary widely, depending on the capacity of the preconditioner, the extruder throughput and/or the desired residence time of the mixture in the preconditioner or extruder barrel. Generally, the speed of the paddles is from about 100 to about 1300 revolutions per minute (rpm). Agitation must be high enough to obtain even hydration and good mixing.

Typically, the protein-containing material is pre-conditioned prior to introduction into the extrusion apparatus by contacting the pre-mix with moisture (i.e., steam and/or water). Preferably the protein-containing material is heated to a temperature of from about 25° C. to about 80° C., more preferably from about 30° C. to about 40° C. in the preconditioner using appropriate water temperatures.

Typically, the protein-containing material pre-mix is conditioned for a period of about 30 to about 60 seconds, depending on the speed and the size of the conditioner. The pre-mix is contacted with steam and/or water and heated in the pre-conditioner at generally constant steam flow to achieve the desired temperatures. The water and/or steam conditions (i.e., hydrates) the pre-mix, increases its density, and facilitates the flowability of the dried mix without interference prior to introduction to the extruder barrel where the proteins are texturized. If low moisture pre-mix is desired, the conditioned pre-mix may contain from about 1% to about 35% (by weight) water. If high moisture pre-mix is desired, the conditioned pre-mix may contain from about 35% to about 80% (by weight) water.

The conditioned pre-mix typically has a bulk density of from about 0.25 g/cm³ to about 0.6 g/cm³. Generally, as the bulk density of the pre-conditioned protein mixture increases within this range, the protein mixture is easier to process.

(iii) Extrusion Process

The conditioned pre-mix is then fed into an extruder to heat, shear, and ultimately plasticize the mixture. The extruder may be selected from any commercially available extruder and may be a single screw extruder or preferably a twin-screw extruder that mechanically shears the mixture with the screw elements.

Whichever extruder is used, it should be run in excess of about 50% motor load. Typically, the conditioned pre-mix is introduced to the extrusion apparatus at a rate of between about 16 kilograms per minute to about 60 kilograms per minute. In another embodiment, the conditioned pre-mix is introduced to the extrusion apparatus at a rate between 20 kilograms per minute to about 40 kilograms per minute. the conditioned pre-mix is introduced to the extrusion apparatus at a rate of between about 26 kilograms per minute to about 32 kilograms per minute. Generally, it has been observed that the density of the extrudate decreases as the feed rate of pre-mix to the extruder increases.

The pre-mix is subjected to shear and pressure by the extruder to plasticize the mixture. The screw elements of the extruder shear the mixture as well as create pressure in the extruder by forcing the mixture forwards though the extruder and through the die. Preferably, the screw motor speed is set to a speed of from about 200 rpm to about 500 rpm, and more preferably from about 300 rpm to about 450 rpm, which moves the mixture through the extruder at a rate of at least about 20 kilograms per minute, and more preferably at least about 40 kilograms per minute. Preferably the extruder generates an extruder barrel exit pressure of from about 50 to about 3000 psig.

The extruder controls the temperature of the mixture as it passes through the extruder denaturing the protein in the mixture. The extruder includes a means for controlling the temperature of the mixture to ensure temperatures of from about 100° C. to about 180° C. Preferably the means for controlling the temperature of the mixture in the extruder comprises extruder barrel jackets into which heating or cooling media such as steam or water may be introduced to control the temperature of the mixture passing through the extruder. The extruder may also include steam injection ports for directly injecting steam into the mixture within the extruder. The extruder preferably includes multiple heating zones that can be controlled to independent temperatures, where the temperatures of the heating zones are preferably set to control the temperature of the mixture as it proceeds through the extruder. For example, the extruder may be set in a four temperature zone arrangement, where the first zone (adjacent the extruder inlet port) is set to a temperature of from about 80° C. to about 100° C., the second zone is set to a temperature of from about 100° C. to 135° C., the third zone is set to a temperature of from 135° C. to about 150° C., and the fourth zone (adjacent the extruder exit port) is set to a temperature of from about 150° C. to about 180° C. The extruder may be set in other temperature zone arrangements, as desired. For example, the extruder may be set in a five temperature zone arrangement, where the first zone is set to a temperature of about 25° C., the second zone is set to a temperature of about 50° C., the third zone is set to a temperature of about 95° C., the fourth zone is set to a temperature of about 130° C., and the fifth zone is set to a temperature of about 150° C.

The mixture forms a melted plasticized mass in the extruder. A die assembly is attached to the extruder in an arrangement that permits the plasticized mixture to flow from the extruder exit port into the die assembly, wherein the die assembly consists of a die and a back plate. Additionally, the die assembly produces substantial alignment of the protein fibers within the plasticized mixture as it flows through the die assembly. The back plate in combination with the die creates at least one central chamber that receives the melted plasticized mass from the extruder through at least one central opening. From the at least one central chamber, the melted plasticized mass is directed by flow directors into at least one elongated tapered channel. Each elongated tapered channel leads directly to an individual die aperture. The extrudate exits the die through at least one aperture in the periphery or side of the die assembly at which point the protein fibers contained within are substantially aligned. It is also contemplated that the extrudate may exit the die assembly through at least one aperture in the die face, which may be a die plate affixed to the die.

The width and height dimensions of the die aperture(s) are selected and set prior to extrusion of the mixture to provide the fibrous material extrudate with the desired dimensions. The width of the die aperture(s) may be set so that the extrudate resembles from a cubic chunk of meat to a steak filet, where widening the width of the die aperture(s) decreases the cubic chunk-like nature of the extrudate and increases the filet-like nature of the extrudate. Preferably the width of the die aperture(s) is/are set to a width of from about 5 millimeters to about 40 millimeters.

The height dimension of the die aperture(s) may be set to provide the desired thickness of the extrudate. The height of the aperture(s) may be set to provide a very thin extrudate or a thick extrudate. Preferably, the height of the die aperture(s) may be set to from about 1 millimeter to about 30 millimeters, and more preferably from about 8 millimeters to about 16 millimeters.

It is also contemplated that the die aperture(s) may be round. The diameter of the die aperture(s) may be set to provide the desired thickness of the extrudate. The diameter of the aperture(s) may be set to provide a very thin extrudate or a thick extrudate. Preferably, the diameter of the die aperture(s) may be set to from about 1 millimeter to about 30 millimeters, and more preferably from about 8 millimeters to about 16 millimeters.

The extrudate can be cut after exiting the die assembly. Suitable apparatuses for cutting the extrudate after it exits the die assembly include flexible knives manufactured by Wenger Manufacturing, Inc. (Sabetha, Kans.) and Clextral, Inc. (Tampa, Fla.). A delayed cut can also be done to the extrudate. One such example of a delayed cut device is a guillotine device.

The dryer, if one is used, generally comprises a plurality of drying zones in which the air temperature may vary. The extrudate will be present in the dryer for a time sufficient to produce an extrudate having the desired moisture content. Thus, the temperature of the air is not important, if a lower temperature is used longer drying times will be required than if a higher temperature is used. Generally, the temperature of the air within one or more of the zones will be from about 100° C. to about 185° C. At such temperatures the extrudate is generally dried for at least about 5 minutes and more generally, for at least about 10 minutes. Suitable dryers include those manufactured by Wolverine Proctor & Schwartz (Merrimac, Mass.), National Drying Machinery Co. (Philadelphia, Pa.), Wenger (Sabetha, Kans.), Clextral (Tampa, Fla.), and Buehler (Lake Bluff, Ill.).

The desired moisture content may vary widely depending on the intended application of the extrudate. Generally speaking, the extruded material has a moisture content of from about 6% to about 13% by weight, if dried. Although not required in order to separate the fibers, hydrating in water until the water is absorbed is one way to separate the fibers. If the protein material is not dried or not fully dried, its moisture content is higher, generally from about 16% to about 30% by weight.

The dried extrudate may further be comminuted to reduce the average particle size of the extrudate. Suitable grinding apparatus include hammer mills such as Mikro Hammer Mills manufactured by Hosokawa Micron Ltd. (England), Fitzmill® manufactured by the Fitzpatrick Company (Elmhurst, Ill.), Comitrol® processors made by Urschel Laboratories, Inc. (Valparaiso, Ind.), and roller mills such as RossKamp Roller Mills manufactured by RossKamp Champion (Waterloo, Ill.).

### (f) Characterization of the Structured Protein Products

The extrudates produced in I(e) typically comprise the structured plant protein products comprising protein fibers that are substantially aligned. In the context of this invention “substantially aligned” generally refers to the arrangement of protein fibers such that a significantly high percentage of the protein fibers forming the structured plant protein product are contiguous to each other at less than approximately a 45° angle when viewed in a horizontal plane. Typically, an average of at least 55% of the protein fibers comprising the structured plant protein product are substantially aligned. In another embodiment, an average of at least 60% of the protein fibers comprising the structured plant protein product are substantially aligned. In a further embodiment, an average of at least 70% of the protein fibers comprising the structured plant protein product are substantially aligned. In an additional embodiment, an average of at least 80% of the protein fibers comprising the structured plant protein product are substantially aligned. In yet another embodiment, an average of at least 90% of the protein fibers comprising the structured plant protein product are substantially aligned. Methods for determining the degree of protein fiber alignment are known in the art and include visual determinations based upon micrographic images. By way of example, FIGS. 1 and 2 depict micrographic images that illustrate the difference between a structured plant protein product having substantially aligned protein fibers compared to a plant protein product having protein fibers that are significantly crosshatched. FIG. 1 depicts a structured plant protein product prepared according to I(a)-I(e) having protein fibers that are substantially aligned. Contrastingly, FIG. 2 depicts a plant protein product containing protein fibers that are significantly crosshatched and not substantially aligned. Because the protein fibers are substantially aligned, as shown in FIG. 1, the structured plant protein products utilized in the invention generally have the texture and consistency of cooked muscle meat. In contrast, extrudates having protein fibers that are randomly oriented or crosshatched generally have a texture that is soft or spongy.

In addition to having protein fibers that are substantially aligned, the structured plant protein products also typically have shear strength substantially similar to whole meat muscle. In this context of the invention, the term “shear strength” provides one means to quantify the formation of a sufficient fibrous network to impart whole-muscle like texture and appearance to the plant protein product. Shear strength is the maximum force in grams needed to shear through a given sample. A method for measuring shear strength is described in Example 1. Generally speaking, the structured plant protein products of the invention will have average shear strength of at least 1400 grams. In an additional embodiment, the structured plant protein products will have average shear strength of from about 1500 to about 1800 grams. In yet another embodiment, the structured plant protein products will have average shear strength of from about 1800 to about 2000 grams. In a further embodiment, the structured plant protein products will have average shear strength of from about 2000 to about 2600 grams. In an additional embodiment, the structured plant protein products will have average shear strength of at least 2200 grams. In a further embodiment, the structured plant protein products will have average shear strength of at least 2300 grams. In yet another embodiment, the structured plant protein products will have average shear strength of at least 2400 grams. In still another embodiment, the structured plant protein products will have average shear strength of at least 2500 grams. In a further embodiment, the structured plant protein products will have average shear strength of at least 2600 grams.

A means to quantify the size of the protein fibers formed in the structured plant protein products may be done by a shred characterization test. Shred characterization is a test that generally determines the percentage of large pieces formed in the structured plant protein product. In an indirect manner, percentage of shred characterization provides an additional means to quantify the degree of protein fiber alignment in a structured plant protein product. Generally speaking, as the percentage of large pieces increases, the degree of protein fibers that are aligned within a structured plant protein product also typically increases. Conversely, as the percentage of large pieces decreases, the degree of protein fibers that are aligned within a structured plant protein product also typically decreases. A method for determining shred characterization is detailed in Example 2. The structured plant protein products of the invention typically have an average shred characterization of at least 10% by weight of large pieces. In a further embodiment, the structured plant protein products have an average shred characterization of from about 10% to about 15% by weight of large pieces. In another embodiment, the structured plant protein products have an average shred characterization of from about 15% to about 20% by weight of large pieces. In yet another embodiment, the structured plant protein products have an average shred characterization of from about 20% to about 50% by weight of large pieces. In another embodiment, the average shred characterization is at least 20% by weight, at least 21% by weight, at least 22% by weight, at least 23% by weight, at least 24% by weight, at least 25% by weight, or at least 26% by weight large pieces.

Suitable structured plant protein products of the invention generally have protein fibers that are substantially aligned, have average shear strength of at least 1400 grams, and have an average shred characterization of at least 10% by weight large pieces. More typically, the structured plant protein products will have protein fibers that are at least 55% aligned, have average shear strength of at least 1800 grams, and have an average shred characterization of at least 15% by weight large pieces. In an exemplary embodiment, the structured plant protein products will have protein fibers that are at least 55% aligned, have average shear strength of at least 2000 grams, and have an average shred characterization of at least 17% by weight large pieces. In another exemplary embodiment, the structured plant protein products will have protein fibers that are at least 55% aligned, have average shear strength of at least 2200 grams, and have an average shred characterization of at least 20% by weight large pieces. In another exemplary embodiment, the structured plant protein products will have protein fibers that are at least 55% aligned, have average shear strength of at least 2400 grams, and have an average shred characterization of at least 20% by weight large pieces

### (II) Animal Meat

The animal meat compositions, in addition to structured plant protein product, also comprise animal meat. By way of example, meat and meat ingredients defined specifically for the various structured vegetable protein patents include intact or ground beef, pork, lamb, mutton, horsemeat, goat meat, meat, fat and skin of poultry (domestic fowl such as chicken, duck, goose or turkey) and more specifically flesh tissues from any fowl (any bird species), fish flesh derived from both fresh and salt water fish such as catfish, tuna, sturgeon, salmon, bass, muskie, pike, bowfin, gar, paddlefish, bream, carp, trout, walleye, snakehead and crappie, animal flesh of shellfish and crustacean origin, animal flesh trim and animal tissues derived from processing such as frozen residue from sawing frozen fish, chicken, beef, pork etc., chicken skin, pork skin, fish skin, animal fats such as beef fat, pork fat, lamb fat, chicken fat, turkey fat, rendered animal fat such as lard and tallow, flavor enhanced animal fats, fractionated or further processed animal fat tissue, finely textured beef, finely textured pork, finely textured lamb, finely textured chicken, low temperature rendered animal tissues such as low temperature rendered beef and low temperature rendered pork, mechanically separated meat or mechanically deboned meat (MDM) (meat flesh removed from bone by various mechanical means) such as mechanically separated beef, mechanically pork, mechanically separated fish, mechanically separated chicken, mechanically separated turkey, any cooked animal flesh and organ meats derived from any animal species. Meat flesh should be extended to include muscle protein fractions derived from salt fractionation of the animal tissues, protein ingredients derived from isoelectric fractionation and precipitation of animal muscle or meat and hot boned meat as well as mechanically prepared collagen tissues and gelatin. Additionally, meat, fat, connective tissue and organ meats of game animals such as buffalo, deer, elk, moose, reindeer, caribou, antelope, rabbit, bear, squirrel, beaver, muskrat, opossum, raccoon, armadillo and porcupine as well as well as reptilian creatures such as snakes, turtles and lizards should be considered meat.

It is also envisioned that a variety of meat qualities may be utilized in the invention depending upon the product's intended use. For example, whole meat muscle that is either, ground or in chunk or steak form may be, utilized. In an additional embodiment, mechanically deboned meat (MDM) may be utilized. In the context of the present invention, “MDM” is a meat paste that is recovered from a variety of animal bones, such as, beef, pork and chicken bones, using commercially available equipment. MDM is generally a comminuted product that is devoid of the natural fibrous texture found in intact muscles. In other embodiments, a combination of MDM and whole meat muscle may be utilized.

### (III) Process for Producing Food Products Comprising Animal Meat and Simulated Animal Meat Compositions

Another aspect of the invention provides a process for producing food products comprising animal meat compositions. An animal meat composition may comprise a mixture of animal meat and structured plant protein product, or it may comprise structured plant protein product. The process generally comprises hydrating the structured plant protein product, reducing its particle size if necessary, optionally flavoring and coloring the structured plant protein product, optionally mixing it with animal meat, and further processing the composition into a food product.

The pH-lowering agent may be added at several stages during the preparation of the composition of the invention. When an animal meat composition is prepared, the pH-lowering agent may be combined with the animal meat to form, a mixture and then the mixture may be combined with the structured plant protein product. Alternatively, the structured plant protein product may be combined with the animal meat to form a mixture and then the mixture may be combined with the pH-lowering agent. In an additional embodiment, the animal meat, structured plant protein product, and pH-lowering agent may all be combined substantially simultaneously. When a simulated meat composition is prepared, the pH-lowering agent may be added prior to extrusion of the plant protein material or it may be added at any stage during the preparation of the composition, as detailed below, such as during hydration, coloring, or before a cooking procedure.

### (a) Hydrating the Structured Plant Protein Product

The structured plant protein product may be mixed with water to rehydrate it. The amount of water added to the structured plant protein product can and will vary. The ratio of water to structured plant protein product may range from about 1.5:1 to about 4:1. In a preferred embodiment, the ration of water to structured plant protein product may be about 2.5:1. As detailed above, the pH-lowering agent may be contacted with the structured plant protein product during the hydration process.

(b) Optionally Blend with Animal Meat

The hydrated structured plant protein product may be blended with animal meat to produce animal meat compositions. Any of the animal meats detailed in II above or otherwise known in the art may be utilized. In general, the structured plant protein product will be blended with animal meat that has a similar particle size. Typically, the amount of structured plant protein product in relation to the amount of animal meat in the animal meat compositions can and will vary depending upon the composition's intended use. By way of example, when a significantly vegetarian composition that has a relatively small degree of animal flavor is desired, the concentration of animal meat in the animal meat composition may be about 45%, 40%, 35%, 30%, 25%, 20%, 15%, 10%, 5%, 2%, or 0% by weight. Alternatively, when an animal meat composition having a relatively high degree of animal meat flavor is desired, the concentration of animal meat in the animal meat composition may be about 50%, 55%, 60%, 65%, 70%, or 75% by weight. Consequently, the concentration of the hydrated structured plant protein product in the animal meat composition may be about 25%, 30%, 35%, 40%, 45%, 50%, 55%, 60%, 65%, 70%, 75%, 80%, 85%, 90%, 95%, or 99% by weight. In one embodiment, the animal meat composition is mixed with the hydrated structured plant protein at a temperature of −2° C. to about 12° C.

Depending upon the food product, the animal meat is typically pre-cooked to partially dehydrate the flesh and prevent the release of those fluids during further processing applications (e.g., such as retort cooking), to remove natural liquids or oils that may have strong flavors, to coagulate the animal protein and loosen the meat from the skeleton, or to develop desirable and textural flavor properties. The pre-cooking process may be carried out in steam, water, oil, hot air, smoke, or a combination thereof. The animal meat is generally heated until the internal temperature is between 60° C. and 85° C. In one embodiment, the animal meat composition is mixed with the hydrated structured plant protein at an elevated temperature corresponding to the temperature of the meat product.

### (c) Optionally Add a Coloring Agent

It is also envisioned that the animal meat composition or simulated meat composition may be combined with a suitable coloring agent such that the color of the composition resembles the color of animal meat it simulates. The compositions of the invention may be colored to resemble dark animal meat or light animal meat. By way of example, the composition may be colored with a natural colorant, a combination of natural colorants, an artificial colorant, a combination of artificial colorants, or a combination of natural and artificial colorants. Suitable examples of natural colorants approved for use in food include annatto (reddish-orange), anthocyanins (red to blue, depends upon pH), beet juice, beta-carotene (orange), beta-APO 8 carotenal (orange), black currant, burnt sugar; canthaxanthin (pink-red), caramel, carmine/carminic acid (bright red), cochineal extract (red), curcumin (yellow-orange); lutein (red-orange); mixed carotenoids (orange), monascus (red-purple, from fermented red rice), paprika, red cabbage juice, riboflavin (yellow), saffron, titanium dioxide (white), and turmeric (yellow-orange). Suitable examples of artificial colorants approved for use in food include FD&C (Food Drug & cosmetics) Red Nos. 3 (carmosine), 4 (fast red E), 7 (ponceau 4R), 9 (amaranth), 14 (erythrosine), 17 (allura red), 40 (allura red AC) and FD&C Yellow Nos. 5 (tartrazine), 6 (sunset yellow) and 13 (quinoline yellow). Food colorants may be dyes, which are powders, granules, or liquids that are soluble in water. Alternatively, natural and artificial food colorants may be lake colors, which are combinations of dyes and insoluble materials. Lake colors are not oil soluble, but are oil dispersible; they tint by dispersion.

The type of colorant or colorants and the concentration of the colorant or colorants will be adjusted to match the color of the animal meat to be simulated. The final concentration of a natural food colorant may range from about 0.01% percent to about 4% by weight.

The color system may further comprise an acidity regulator to maintain the pH in the optimal range for the colorant. The acidity regulator may be an acidulent. Examples of acidulents that may be added to food include citric acid, acetic acid (vinegar), tartaric acid, malic acid, fumaric acid, lactic acid, phosphoric acid, sorbic acid, and benzoic acid. The final concentration of the acidulent in an animal meat composition may range from about 0.001% to about 5% by weight. The final concentration of the acidulent may range from about 0.01% to about 2% by weight. The final concentration of the acidulent may range from about 0.1% to about 1% by weight. The acidity regulator may also be a pH-raising agent, such as disodium diphosphate.

### (d) Addition of Optional Ingredients

The simulated animal meat compositions or the compositions blended with animal meat may optionally include a variety of flavorings, spices, antioxidants, or other ingredients to nutritionally enhance the final food product. As will be appreciated by a skilled artisan, the selection of ingredients added to the animal meat composition can and will depend upon the food product to be manufactured.

The animal meat compositions or simulated animal meat compositions may further comprise an antioxidant. The antioxidant may prevent the oxidation of the polyunsaturated fatty acids (e.g., omega-3 fatty acids) in the animal meat, and the antioxidant may also prevent oxidative color changes in the colored structured plant protein product and the animal meat. The antioxidant may be natural or synthetic. Suitable antioxidants include, but are not limited to, ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, ice bran extract, lactic acid and its salts, lecithin, lecithin citrate; R-alpha-lipoic acid, lutein, lycopene, malic, acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., Ionox 100), 2,4-(tris-3′,5′-bi-tert-butyl-4′-hydroxybenzyl)-mesitylene (i.e., Ionox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof. The concentration of an antioxidant in an animal meat composition may range from about 0.0001% to about 20% by weight. In another embodiment, the concentration of an antioxidant in an animal meat composition may range from about 0.001% to about 5% by weight. In yet another embodiment, the concentration of an antioxidant in an animal meat composition may range from about 0.01% to about 1% by weight.

In an additional embodiment, the animal meat compositions or simulated animal meat compositions may further comprise a flavoring agent such as an animal meat flavor, an animal meat oil, spice extracts, spice oils, natural smoke solutions, natural smoke extracts, yeast extract, and shiitake extract. Additional flavoring agents may include onion flavor, garlic flavor, or herb flavors. The animal meat composition may further comprise a flavor enhancer. Examples of flavor enhancers that may be used include salt (sodium chloride), glutamic acid salts (e.g., monosodium glutamate), glycine salts, guanylic acid salts, inosinic acid salts, 5′-ribonucleotide salts, hydrolyzed proteins, and hydrolyzed vegetable proteins.

In an additional embodiment, the animal meat compositions may further comprise a thickening or a gelling agent, such as alginic acid and its salts, agar, carrageenan and its salts, processed Eucheuma seaweed, gums (carob bean, guar, tragacanth, and xanthan), pectins, sodium carboxymethylcellulose, and modified starches.

In a further embodiment, the animal meat compositions may further comprise a nutrient such as a vitamin, a mineral, an antioxidant, an omega-3 fatty acid, or an herb. Suitable vitamins include Vitamins A, C, and E, which are also antioxidants, and Vitamins B and D. Examples of minerals that may be added include the salts of aluminum, ammonium, calcium, magnesium, and potassium. Suitable omega-3 fatty acids include docosahexaenoic acid (DHA). Herbs that may be added include basil, celery leaves, chervil, chives, cilantro, parsley, oregano, tarragon, and thyme.

### (e) Variety of Food Products

The animal meat compositions may be processed into a variety of food product for either human or animal consumption. By way of non-limiting example, the final product may be an animal meat composition for human consumption that simulates a ground meat product, a steak product, a sirloin tip product, a kebab product, a shredded product, a chunk meat product, or a nugget product. Any of the foregoing products may be placed in a tray with overwrap, vacuum packed, retort canned or pouched, or frozen.

It is also envisioned that the animal compositions of the present invention may be utilized in a variety of animal diets. In one embodiment, the final product may be an animal meat composition formulated for companion animal consumption. In another embodiment, the final product may be an animal meat composition formulated for agricultural or zoo animal consumption. A skilled artisan can readily formulate the meat compositions for use in companion animal, agricultural animal or zoo animal diets.

### DEFINITIONS

The term “extrudate” as used herein refers to the product of extrusion. In this context, the structured plant protein products comprising protein fibers that are substantially aligned may be extrudates in some embodiments.

The term “fiber” as used herein refers to a structured>plant protein product having a size of approximately 4 centimeters in length and 0.2 centimeters in width after the shred characterization test detailed in Example 2 is performed. Fibers generally form Group 1 in the shred characterization test as described in Example 2. In this context, the term “fiber” does not include the nutrient class of fibers, such as soybean cotyledon fibers, and also does not refer to the structural formation of substantially aligned protein fibers comprising the plant protein products.

The term “animal meat” as used herein refers to the flesh, whole meat muscle, or parts thereof derived from an animal.

The term “gluten” as used herein refers to a protein fraction in cereal grain flour, such as wheat, that possesses a high content of protein as well as unique structural and adhesive properties.

The term “gluten free starch” as used herein refers to modified tapioca starch. Gluten free or substantially gluten free starches are made from wheat, corn, and tapioca based starches. They are gluten free because they do not contain the gluten from wheat, oats, rye or barley.

The term “large piece” as used herein is the manner in which a structured plant protein product's shred percentage is characterized. The determination of shred characterization is detailed in Example 2.

The term “protein fiber” as used herein refers the individual continuous filaments or discrete elongated pieces of varying lengths that together define the structure of the plant protein products of the invention. Additionally, because the plant protein products of the invention have protein fibers that are substantially aligned, the arrangement of the protein fibers impart the texture of whole meat muscle to the plant protein products.

The term “simulated” as used herein refers to an animal meat composition that contains no animal meat.

The term “soy cotyledon fiber” as used herein refers to the polysaccharide portion of soy cotyledons containing at least about 70% dietary fiber. Soy cotyledon fiber typically contains some minor amounts of soy protein, but may also be 100% fiber. Soy cotyledon fiber, as used herein, does not refer to, or include, soy hull fiber. Generally, soy cotyledon fiber is formed from soybeans by removing the hull and germ of the soybean, flaking or grinding the cotyledon and removing oil from the flaked or ground cotyledon, and separating the soy cotyledon fiber from the soy material and carbohydrates of the cotyledon.

The term “soy protein concentrate” as used herein is a soy material having a protein content of from about 65% to less than about 90% soy protein on a moisture-free basis. Soy protein concentrate also contains soy cotyledon fiber, typically from about 3.5% up to about 20% soy cotyledon fiber by weight on a moisture-free basis. A soy protein concentrate is formed from soybeans by removing the hull and germ of the soybean, flaking or grinding the cotyledon and removing oil from the flaked or ground cotyledon, and separating the soy protein and soy cotyledon fiber from the soluble carbohydrates of the cotyledon.

The term “soy flour” as used herein, refers to a comminuted form of defatted soybean material, preferably containing less than about 1% oil, formed of particles having a size such that the particles can pass through a No. 100 mesh (U.S. Standard) screen. The soy cake, chips, flakes, meal, or mixture of the materials are comminuted into soy flour using conventional soy grinding processes. Soy flour has a soy protein content of about 49% to about 65% on a moisture free basis.

The term “soy protein isolate” as used herein is a soy material having a protein content of at least about 90% soy protein on a moisture free basis. A soy protein isolate is formed from soybeans by removing the hull and germ of the soybean from the cotyledon, flaking or grinding the cotyledon and removing oil from the flaked or ground cotyledon, separating the soy protein and carbohydrates of the cotyledon from the cotyledon fiber, and subsequently separating the soy protein from the carbohydrates.

The term “strand” as used herein refers to a structured plant protein product having a size of approximately 2.5 to about 4 centimeters in length and greater than approximately 0.2 centimeter in width after the shred characterization test detailed in Example 2 is performed. Strands generally form Group 2 as defined in Example 2 in the shred characterization test.

The term “starch” as used herein refers to starches derived from any native source. Typically sources for starch are cereals, tubers, roots, legumes, and fruits.

The term “wheat flour” as used herein refers to flour obtained from the milling of wheat. Generally speaking, the particle size of wheat flour is from about 14 μm to about 120 μm.

### EXAMPLES

Examples 1-9 illustrate various embodiments of the invention.

### Example 1

### Determination of Shear Strength

Shear strength of a sample is measured in grams and may be determined by the following procedure. Weigh a sample of the structured plant protein product and place it in a heat sealable pouch and hydrate the sample with approximately three times the sample weight of room temperature tap water. Evacuate the pouch to a pressure of about 0.01 bar and seal the pouch. Permit the sample to hydrate for about 12 to about 24 hours. Remove the hydrated sample and place it on the texture analyzer base plate oriented so that a knife from the texture analyzer will cut through the diameter of the sample. Further, the sample should be oriented under the texture analyzer knife such that the knife cuts perpendicular to the long axis of the textured piece. A suitable knife used to cut the extrudate is a model TA-45, incisor blade manufactured by Texture Technologies (USA). A suitable texture analyzer to perform this test is a model TA, TXT2 manufactured by Stable Micro Systems Ltd. (England) equipped with a 25, 50, or 100 kilogram load. Within the context of this test, shear strength is the maximum force in grams needed to shear through the sample.

### Example 2

### Determination of Shred Characterization

A procedure for determining shred characterization may be performed as follows. Weigh about 150 grams of a structured plant protein product using whole pieces only. Place the sample into a heat-sealable plastic bag and add about 450 grams of water at 25° C. Vacuum seal the bag at about 150 mm Hg and allow the contents to hydrate for about 60 minutes. Place the hydrated sample in the bowl of a Kitchen Aid mixer model KM14G0 equipped with a single blade paddle and mix the contents at 130 rpm for two minutes. Scrape the paddle and the sides of the bowl, returning the scrapings to the bottom of the bowl. Repeat the mixing and scraping two times. Remove ˜200 g of the mixture from the bowl. Separate the ˜200 g of mixture into one of two groups. Group 1 is the portion of the sample having fibers at least 4 centimeters in length, and at least 0.2 centimeters wide. Group 2 is the portion of the sample having strands between 2.5 cm and 4.0 cm long, and which are 0.2 cm wide. Weigh each group, and record the weight. Add the weight of each group together, and divide by the starting weight (e.g. ˜200 g). This determines the percentage of large pieces in the sample. If the resulting value is below 15%, or above 20%, the test is complete. If the value is between 15% and 20%, then weigh out another ˜200 g from the bowl, separate the mixture into groups one and two, and perform the calculations again.

### Example 3

### Production of Structured Plant Protein Products

The following extrusion process may be used to prepare the structured plant protein products of the invention, such as the soy structured plant protein products utilized in Examples 1 and 2. Added to a dry blend mixing tank are the following: 1000 kilograms (kg) Supro® 620 (soy isolate), 440 kg wheat gluten, 171 kg wheat starch, 34 kg soy cotyledon fiber, 9 kg dicalcium phosphate, and 1 kg L-cysteine. The contents are mixed to form a dry blended soy protein mixture. The dry blend is then transferred to a hopper from which the dry blend is introduced into a preconditioner along with 480 kg of water to form a conditioned soy protein pre-mixture. The conditioned soy protein pre-mixture is then fed to a twin-screw extrusion apparatus (Wenger Model TX-168 extruder by Wenger Manufacturing, Inc. (Sabetha, Kans.)) at a rate of not more than 25 kg/minute. The extrusion apparatus comprises five temperature control zones, with the protein mixture being controlled to a temperature of from about 25° C. in the first zone, about 50° C. in the second zone, about 95° C. in the third zone, about 130° C. in the fourth zone, and about 150° C. in the fifth zone. The extrusion mass is subjected to a pressure of at least about 400 psig in the first zone up to about 1500 psig in the fifth zone. Water, 60 kg per hour, is injected into the extruder barrel, via one or more injection jets in communication with a heating zone. The molten extruder mass exits the extruder barrel through a die assembly consisting of a die and a backplate. As the mass flows through the die assembly the protein fibers contained within are substantially aligned with one another forming a fibrous extrudate. As the fibrous extrudate exits the die assembly, it is cut with flexible knives and the cut mass is then dried to a moisture content of about 10% by weight.

### Example 4

### Production of Structured Plant Protein Products with Adjusted pH

The following extrusion process may be used to prepare the structured plant protein products with a reduced pH of the invention, such as the soy structured plant protein products utilized in Examples 1 and 2. Added to a dry blend mixing tank are the following: 1000 kilograms (kg) Supro® 620 (soy isolate), 440 kg wheat gluten, 171 kg wheat starch, 34 kg soy cotyledon fiber, 9 kg dicalcium phosphate, and 1 kg L-cysteine. In addition, an amount of a pH modifying agent, such as citric acid (CA) or sodium carbonate (SC) was added during the dry blending. Example pH values are demonstrated below in Table 1. The contents are mixed to form a dry blended soy protein mixture. The dry blend is then transferred to a hopper from which the dry blend is introduced into a preconditioner along with 480 kg of water to form a conditioned soy protein pre-mixture. The conditioned soy protein pre-mixture is then fed to a twin-screw extrusion apparatus (Wenger Model TX-168 extruder by Wenger Manufacturing, Inc. (Sabetha, Kans.)) at a rate of not more than 25 kg/minute. The extrusion apparatus comprises five temperature control zones, with the protein mixture being controlled to a temperature of from about 25° C. in the first zone, about 50° C. in the second zone, about 95° C. in the third zone, about 130° C. in the fourth zone, and about 150° C. in the fifth zone. The extrusion mass is subjected to a pressure of at least about 400 psig in the first zone up to about 1500 psig in the fifth zone. Water, 60 kg per hour, is injected into the extruder barrel, via one or more injection jets in communication with a heating zone. The molten extruder mass exits the extruder barrel through a die assembly consisting of a die and a backplate. As the mass flows through the die assembly the protein fibers contained within are substantially aligned with one another forming a fibrous extrudate. As the fibrous extrudate exits the die assembly, it is cut with flexible knives and the cut mass is then dried to a moisture content of about 10% by weight.

TABLE 1
amount of pH modifying agents by percentage weight related
to post extrusion pH value for structured plant protein.

| | pH modifying agent | |
|---|---|---|
| Plant Protein % by weight | and % by weight | pH post extrusion |
| 100% | None | 6.75 |
| 99.70% | CA - 0.30% | 6.49 |
| 99.10% | CA - 0.90% | 6.03 |
| 98.30% | CA - 1.70% | 5.51 |
| 97.20% | CA - 2.80% | 5.00 |
| 99.80% | SC - 0.20% | 7.05 |
| 99.40% | SC - 0.60% | 7.48 |
| 98.90% | SC - 1.10% | 8.01 |
| 98.40% | SC - 1.60% | 8.54 |

### Example 5

### Comparison of the Texture of Animal Meat Compositions Produced at Different pH Values

To create an animal meat composition with a fibrous, more meat-like texture and appearance, a strategy was devised to produce the composition at the pH level found in rigor meat. When beef, pork, or poultry animals are slaughtered, oxygen becomes limiting and anaerobic metabolism results in the conversion of glycogen to lactic acid with an accompanied reduction in pH. Prior to slaughter, muscle tissue is in the neutral pH range. After slaughter, pH typically drops to about 5.4 to 5.8, and the drop is, due to the accumulation of lactic acid in the muscle tissue. Lactic acid was chosen as a pH-lowering agent since it is naturally occurring in muscle tissue after slaughter. The lactic acid used in Treatment 2 is PURAC® FCC 88 (Purac America, Lincolnshire, Ill. 60069) lowers the pH to within the level of 5.4 to 5.8 as would be found in post rigor meat. To test the affect of the pH-lowering agent the meat blend in Treatment 1 does not include an amount of lactic acid; conversely, the meat blend in Treatment 2 does include an amount of lactic acid.

The animal meat composition blends were prepared identically, except for the addition of the pH-lowering agent (lactic acid). For each the following ingredients were mixed at 3-4° C. The list and percentage by weight of the ingredients follows in Table 2. The tempered chicken MDM was ground to 6.35 mm and the beef was ground to 3.175 mm prior to blending. The SUPRO®MAX 5050 (plant protein product) was placed into the single paddle mixer (Model AV50, Talleres Cato, s.a., Spain) to hydrate with water for 20 minutes while shredding under vacuum. The chicken MDM, beef, sodium nitrite and salt were then added to the shredded SUPRO®MAX 5050 and vacuum mixed for 10 minutes. All the remaining ingredients were then added to the mixer and mixed for 5 minutes under vacuum. At this stage the pH of Treatment 2 was lowered to 5.6 by the addition of the PURAC® FCC 88 lactic acid. The pH of the Treatment 1 blend was not adjusted. The meat blend was then formed into patties using a Hollymatic forming machine (Hollymatic Corporation, Countryside, Ill.). All the patties were then cooked to an internal temperature of 71° C. at 177° C. in a Combo Oven (Groen Combination Steamer Oven, Model CC20-E Convection Combo, Groen, Jackson, Miss.) with the convection heat and steam combination option selected. All products were then frozen for storage prior to further testing. Prior to texture and shear analysis samples were brought to room temperature, approximately 23° C.

TABLE 2
Formulations for the meat blends.

| | Treatment 1 | Treatment 2 |
|---|---|---|
| Ingredients | Percent | Percent |
| Beef 90/10 | 5.000 | 5.000 |
| Chicken MDM 18% | 45.000 | 45.000 |
| Water/Ice | 31.390 | 30.990 |
| SUPRO ® MAX 5050 | 10.000 | 10.000 |
| SUPRO ® EX 32 | 6.000 | 6.000 |
| Sodium Nitrite | 0.015 | 0.015 |
| Sodium | 0.100 | 0.100 |
| Tripolyphosphate | | |
| Salt | 0.050 | 0.050 |
| Sodium Acid | 1.000 | 1.000 |
| Pyrophosphate | | |
| Spices | 0.930 | 0.930 |
| Erythorbate | 0.045 | 0.045 |
| Caramel color, DD | 0.350 | 0.350 |
| Williamson | | |
| Red Rice Color | 0.120 | 0.120 |
| Lactic Acid (88%) | 0.000 | 0.400 |
| Total | 100.000 | 100.000 |

The pH of the two products was recorded throughout processing by combining 20 g of each Treatment test product with 180 g of distilled water in an Oster® blender for 15 seconds on high and measuring the pH with an Orion pH meter (Model 410A). The pH of treatment 2 was lowered to a post rigor meat pH level. The results from these pH measurements are in Table 3.

TABLE 3
pH of treatments at different stages of the test
product production process.

| | | Cooked Pattie | Pattie pH Following |
|---|---|---|---|
| | Raw Blend pH | pH | Freezing |
| Treatment 1 | 6.33 | 6.58 | 6.60 |
| Treatment 2 | 5.63 | 5.82 | 5.83 |

The texture of the final products was analyzed by 5-bladed Kramer Shear Cell and Texture Profile Analysis (TPA) using the 100 mm round platen at 60% compression with the TA-HDi Texture Analyser (Stable Micro Systems, Ltd., Surrey, UK) with the samples at 25° C. Results of these measurements are shown in Table 4.

TABLE 4
Textural properties of patties. Means with like superscripts
are not significantly different.

| | | Treatment 1 | Treatment 2 |
|---|---|---|---|
| Kramer | Peak Force, g | 34633^{a} | 33169^{a} |
| Shear | Area Under Curve | 105839^{a} | 210007^{b} |
| TPA | Hardness | 37724^{b} | 33086^{a} |
| | Springiness | 0.70050^{a} | 0.69747^{a} |
| | Cohesiviness | 0.56043^{b} | 0.49707^{a} |
| | Gumminess | 21165^{b} | 16447^{a} |
| | Chewiness | 14832^{b} | 11487^{a} |
| | Resilience | 0.22200^{b} | 0.13067^{a} |

As Table 4 demonstrates the patties from Treatment 1 and Treatment 2 are distinguishable. FIGS. 5a and 5b demonstrate that the area under the curve, or the amount of work it took to reach the same force value was significantly different showing a difference between the Treatment 1 and Treatment 2 meat blend.

Further, TPA measurements revealed significant differences in hardness, cohesiveness, gumminess, chewiness and resilience between the two treatments. TPA figures are displayed in FIGS. 6a and 6b to show the texture differences in the two treatments. These differences demonstrate the textural difference found in the meat product when a pH-lowering agent was added to the blend during mixing.

### Example 6

### Comparison of the Shear Value of Simulated Meat Compositions Produced at Different pH Values

Testing was completed to show the hydrated structured plant protein piece alone could be altered in texture by the use of acids, thus demonstrating textural differences found in the structured plant protein piece when a pH-modifying agent was added during the creation of the hydrated structured plant protein. To test this, SUPRO®MAX 5053 (Solae, LLC (St. Louis, Mo.)) pieces were hydrated in a solution of distilled water with differing dilutions of 55% citric acid solution under static vacuum for more than 1 hour. Pieces were then placed into tuna cans with distilled water. These cans were sealed and retorted at 118.3° C. for 75 min. The cans were then cooled in an ice water bath and held at refrigeration temperatures until samples were ready for texture and shear analysis. Prior to texture and shear analysis samples were brought to room temperature, approximately 23° C.

Prior to retorting the pH of each treatment was measured by mixing 20 g of SUPRO®MAX 5053 piece with 180 g of distilled water in an Oster® blender for about 15 seconds. The pH of this was then measured using the Orion pH meter (Model 410A). The same process was used to measure the pH of the piece following retorting and cooling. These measurements can be found in Table 5.

TABLE 5
Measurements of the pH of the SUPRO ® MAX 5053
piece pre-retort and post retort.

| Treatment | Pre-retort pH | Post retort pH |
|---|---|---|
| A | 6.74 | 6.39 |
| B | 5.99 | 5.96 |
| C | 5.46 | 5.48 |
| D | 5.39 | 5.00 |
| E | 4.40 | 4.45 |
| F | 4.04 | 4.05 |

The texture of the treatments was measured by using the TA-45 Incisor knife on the TA-HDi Texture Analyser (Stable Micro Systems, Ltd., Surrey, UK) with the samples at 25° C. The probe measured the shear force in grams needed to shear the SUPRO®MAX 5053 piece. Textural data can be found in Table 6.

TABLE 6
Textural properties of retorted SUPRO ® MAX 5053
pieces as related to pH. Means with like superscripts
are not significantly different.

| Treatment | Shear Force, g | Area Under Curve |
|---|---|---|
| A | 611.9^{c} | 2700^{c} |
| B | 1002.3^{b} | 4098^{b} |
| C | 1415.7^{a} | 6020^{a} |
| D | 1460.7^{a} | 6320^{a} |
| E | 1324.1^{a} | 5150^{ab} |
| F | 1334.2^{a} | 5543^{a} |

Shear force values were different for pH levels of 5.96 to 6.39 compared to 4.05 to 5.48. FIGS. 7a and 7b show shear analysis for two of the treatments and show the textural difference between different pH treatments (treatment A at 6.39 pH vs. treatment C at 5.48 pH). As the table and figures demonstrate the addition of a pH-lowering agent affected the texture of the structured plant protein piece. Specifically, the shear force is not significantly different among Treatments C—F, but Treatments C—F are significantly different that Treatments A-B. Thus demonstrating a significant difference among meat blends with a pH of 6 and above when compared to meat blends with a pH below 6.

### Example 7

### Comparison of the Simulated Meat Compositions Produced at Different pH Values

In this example a strategy was devised to create a meat composition with a fibrous, more meat-like texture and appearance using a hydrated structured plant protein with varying pH values. The animal meat composition blends were prepared similar, and as previously described in Example 5 except the hydrated structured plant protein ingredients were created similar to Example 3 with varying pH levels. For each the following ingredients were mixed at 3-4° C. The list and percentage by weight of the ingredients follows in Table 7. The beef was ground to 3 mm prior to blending. The SUPRO®MAX 5050 was placed into the single paddle mixer (Model AV50, Talleres Cato, s.a., Spain) to hydrate with water for 20 minutes while shredding under vacuum. The beef and flavoring agent (Givaudan Flavors Corporation) were then added to the shredded SUPRO®MAX 5050 and vacuum mixed for 10 minutes. The SUPRO®MAX 5050 ingredient had varying pH levels for each treatment to create the varying pH levels for the meat blend as demonstrated in Table 8. All the remaining ingredients were then added to the mixer and mixed for 5 minutes under vacuum. The amount of pH-adjusting material used to create the SUPRO®MAX 5050 ingredient was dependent on the end pH result desired. The meat blend was then formed into patties using a Hollymatic forming machine (Hollymatic Corporation, Countryside, Ill.). All the patties were then cooked to an internal temperature of 71° C. at 177° C. in a Combo Oven (Groen Combination Steamer Oven, Model CC20-E Convection Combo, Groen, Jackson, Miss.) with the convection heat and steam combination option selected. All products were then frozen for storage prior to further testing.

The pH of the Treatments (meat blends) were recorded by combining 20 g of each Treatment test product with 180 g of distilled water in an Oster® blender for 15 seconds on high and measuring the pH with an Orion pH meter (Model 410A). The results from these pH measurements are in Table 8.

TABLE 7
Formulations for the meat blends.

| | Treatments | Treatments | Control - |
|---|---|---|---|
| | T1-T8 | T1-T8 | All Meat |
| Ingredients | Content % | Content kg | Content % |
| Beef 90/10 | 35.8000 | 1.7900 | 48.800 |
| Beef 70/30 | 21.4000 | 1.0700 | 50.400 |
| Water/Ice | 30.0000 | 1.5000 | 0 |
| SUPRO ® MAX 5050 | 10.000 | 0.5000 | 0 |
| Salt | 0 | 0 | 0.6000 |
| Herbalox | 0 | 0 | 0.2000 |
| Flavoring (Givaudan | 2.8000 | 0.1400 | 0 |
| Flavor # 3005760) | | | |
| pH-adjusting agent | varied | varied | 0 |
| Total | 100.000 | 5.000 | 100.000 |

TABLE 8
Shear analysis and cooking yields of meat blends as related to
pH values of Hydrated Structured Plant Protein compositions.

| | | Cook Yields | |
|---|---|---|---|
| | | (percentage | |
| | Meat Blend | by weight of | Shear Force |
| Treatment | pH | precooked) | (grams) |
| T1 | 6.19 | 81.3% | 11915.94 |
| T2 | 5.64 | 80.4% | 11202.8 |
| T3 | 5.84 | 81.7% | 12638.72 |
| T4 | 6.04 | 79.0% | 12699.48 |
| T5 | 6.19 | 80.1% | 12099.59 |
| T6 | 6.27 | 81.5% | 11670.84 |
| T7 | 6.49 | 81.8% | 11756.88 |
| T8 | 6.51 | 83.1% | 11546.96 |
| Control (all | 5.90 | 74.6% | 15890.64 |
| meat) | | | |

Results of the cooking yield and shear analysis are demonstrated in FIGS. 8 and 9 respectively. The texture of the treatments was measured by using the TA-45 Incisor knife on the TA-HDi Texture Analyser (Stable Micro Systems, Ltd., Surrey, UK) with the samples at 25° C. The probe measured the shear force in grams needed to shear the SUPRO®MAX 5050 piece. Textural data can be found in FIG. 8. The control or all meat product produced a peak force (shear strength) of 15,890. As the figure demonstrates the pH has an affect on the texture of the meat product.

The percentage cook yield measured the percentage weight of the cooked meat product compared to the uncooked weight. As shown, the cooking yields for the meat products are relatively similar, typically in the 80.0% yield. The cooked weight data can be found in FIG. 9. The control or all meat product produced a cooked weight percentage of 74.6%.

### Example 8

### Comparison of the Hydrated Structured Plant Protein Compositions at Different pH Values

The hydrated structured plant protein compositions were prepared according to the steps used in Example 4. Varying amount of pH modifying ingredients, such as sodium carbonate and citric acid, were used to obtain the desired pH level for the hydrated structured plant protein. Table 9 demonstrates the hydrated structured plant protein composition pH levels and the corresponding, shear force, shred test, and chunk density associated with each. The shear analysis was conducted according to the steps outlined in Example 1. The shred analysis was conducted according to the steps outlined in Example 2.

TABLE 9
Shear, Shred, and Chunk analysis of Hydrated Structured Plant Protein
Compositions as related to pH.

| | | | | | Shear | Shred | Chunk |
|---|---|---|---|---|---|---|---|
| | Formulated | | Blend | Chunk | force | (% | density |
| Treatment | pH | pH agent | pH | pH | (grams) | acceptable) | (g/cc) |
| Control | N/A | N/A | 6.72 | 7.03 | 1676 | 17.24 | 0.333 |
| 1 | 5 | CA | 5.09 | 5.39 | 1610 | 6.10 | 0.346 |
| 2 | 5.5 | CA | 5.69 | 5.85 | 1910 | 7.55 | 0.374 |
| 3 | 6 | CA | 6.14 | 6.39 | 1572 | 13.50 | 0.386 |
| 4 | 6.5 | CA | 6.68 | 6.85 | 2160 | 38.94 | 0.322 |
| 5 | 7 | SC | 6.91 | 7.16 | 2252 | 33.84 | 0.343 |
| 6 | 7.5 | SC | 7.45 | 7.95 | 2119 | 31.36 | 0.451 |
| 7 | 8 | SC | 8.20 | 8.97 | 2167 | 38.50 | 0.433 |

* Citric Acid (CA)
** Sodium Carbonate (SC)

As the information demonstrates, the lower the pH of the hydrated structured plant protein the lower the shear force, shred percentage acceptable, and chunk density.

### Example 9

### Comparison of the Hydrated Structured Plant Protein Compositions at Different pH Values

The hydrated structured plant protein compositions were prepared according to the steps used in Example 4. Varying amounts of a pH modifying ingredients, such as sodium carbonate and sodium citrate, were used to obtain the desired pH level for the hydrated structure plant protein. Table 11 and 12 demonstrates those pH levels and the corresponding shear force in grams. The shear analysis was conducted according to the steps described in Example 1 and 7 above.

TABLE 10
Formulations for the Hydrated Structured Plant Protein Compositions

| | | Sodium | Sodium | | Dry |
|---|---|---|---|---|---|
| | Water | Carbonate | Citrate | pH | ingredient |
| Treatment | (grams) | (grams) | (grams) | solution | (grams) |
| T1-H | 499.00 | 1.00 | 0 | 11.0 | 156 |
| T2-I | 499.25 | 0.75 | 0 | 10.8 | 148 |
| T3-J | 499.50 | 0.50 | 0 | 10.9 | 149 |
| T4-K | 499.75 | 0.25 | 0 | 10.5 | 158 |
| T5-L | 500.00 | 0 | 0 | 7.1 | 158 |
| T6-M | 499.75 | 0 | 0.25 | 8.0 | 156 |
| T7-N | 499.50 | 0 | 0.50 | 8.1 | 159 |
| T8-O | 499.25 | 0 | 0.75 | 7.9 | 160 |
| T9-P | 499.00 | 0 | 1.00 | 7.9 | 156 |

TABLE 11
Shear analysis of Hydrated Structured Plant Protein
compositions as related to pH.

| | Hydrated Structured | |
|---|---|---|
| | Vegetable Protein piece | Shear Force |
| Treatment | pH | (grams) |
| T1-H | 7.10 | 1655 |
| T2-I | 6.77 | 1828 |
| T3-J | 6.65 | 2182 |
| T4-K | 6.62 | 2264 |
| T5-L | 6.52 | 2169 |
| T6-M | 6.57 | 2510 |
| T7-N | 6.56 | 2278 |
| T8-O | 6.58 | 2291 |
| T9-P | 6.51 | 2171 |

TABLE 12
Shear Analysis of Hydrated Structured Plant Protein Compositions as
Related to pH.

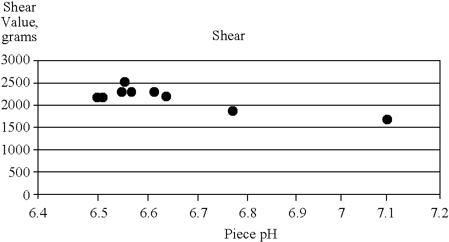

While the invention has been explained in relation to exemplary embodiments, it is to be understood that various modifications thereof will become apparent to those skilled in the art upon reading the description. Therefore, it is to be understood that the invention disclosed herein is intended to cover such modifications as fall within the scope of the following claims.

What is claimed is:

1. A process for producing a structured plant protein product, the process comprising:
(a) combining a plant protein material with a pH-lowering agent to form a mixture, the mixture having a pH below approximately 6.0; and,
(b) extruding the mixture under conditions of elevated temperature and pressure to form a structured plant protein product comprising protein fibers that are substantially aligned.

2. The process of claim 1, wherein the structured plant protein product has an average shear strength of at least 1400 grams and an average shred characterization of at least 10%.

3. The process of claim 2, wherein the structured plant protein product comprises protein fibers substantially aligned in the manner depicted in the micrographic image of FIG. 1.

4. The process of claim 1, wherein the pH-lowering agent is an acid selected from the group consisting of acetic, lactic, hydrochloric, phosphoric, citric, tartaric, malic, and mixtures thereof, wherein the amount of pH-lowering agent combined with the plant protein material is from about 0.1% to about 5% by weight on a dry matter basis.

5. The process of claim 4, wherein the plant protein material is selected from the group consisting of legumes, corn, peas, canola, sunflowers, sorghum, rice, amaranth, potato, tapioca, arrowroot, canna, lupin, rape, wheat, oats, rye, barley, and mixtures thereof.

6. The process of claim 5, further comprising combining at least one animal protein material with the mixture, wherein the animal protein material is selected from the group consisting of casein, caseinates, whey protein, milk protein concentrate, milk protein isolate, ovalbumin, ovoglobulin, ovomucin, ovomucoid, ovotransferrin, ovovitella, ovovitellin, albumin globulin, vitellin, and mixtures thereof.

7. The process of claim 1, wherein the plant protein material has from about 40% to about 90% protein on a dry matter basis.

8. The process of claim 1, wherein the plant protein material comprises protein, starch, gluten, and fiber material comprising:
(a) from about 35% to about 65% soy protein on a dry matter basis;
(b) from about 20% to about 30% wheat gluten on a dry matter basis;
(c) from about 10% to about 15% wheat starch on a dry matter basis; and
(d) from about 1% to about 5% fiber on a dry matter basis.

9. The process of claim 8, wherein the plant protein material further comprises dicalcium phosphate, L-cysteine, and mixtures thereof.

10. A process for producing an animal meat composition, the process comprising:
(a) combining animal meat;
(b) a structured plant protein product comprising protein fibers that are substantially aligned, the structured plant protein product comprising an extrudate of plant protein material;
(c) a pH-lowering agent from about 0.1% to about 5% by weight such that the animal meat composition has a pH below, approximately 6.0; and,
(d) extruding the mixture under conditions of elevated temperature and pressure to form the animal meat composition.

11. The process of claim 10, wherein the structured plant protein product has an average shear strength of at least 1400 grams and an average shred characterization of at least 10%.

12. The process of claim 11, wherein the structured plant protein product comprises protein fibers substantially aligned in the manner depicted in the micrographic image of FIG. 1.

13. The animal meat composition of claim 10, wherein the animal meat and pH-lowering agent are combined to form a mixture, and then the mixture is combined with the structured plant protein product.

14. The animal meat composition of claim 10, wherein the structured plant protein product and pH-lowering agent are combined to form a mixture, and then the mixture is combined with the animal meat.

15. The animal meat composition of claim 10, wherein the structured plant protein product and animal meat are combined to form a mixture, and then the mixture is combined with the pH-lowering agent.

16. The animal meat composition of claim 10, further comprising combining an additional animal protein material with the mixture, wherein the animal protein material is selected from the group consisting of casein, caseinates, whey protein, milk protein concentrate, milk protein isolate, ovalbumin, ovoglobulin, ovomucin, ovomucoid, ovotransferrin, ovovitella, ovovitellin, albumin globulin, vitellin, and mixtures thereof.

17. An animal meat composition, the animal meat composition comprising:
(a) animal meat;
(b) a structured plant protein product comprising protein fibers that are substantially aligned, the structured plant protein product comprising an extrudate of plant protein material; and
(c) a pH-lowering agent in an amount such that the animal meat composition has a pH below approximately 6.0.

18. The animal meat composition of claim 17, wherein the concentration of structured plant protein product present in the animal meat composition ranges from about 25% to about 99% by weight, the concentration of animal meat present ranges from about 1% to about 75% by weight; and the concentration of pH-lowering agent ranges from about 0.1% to about 5% by weight.

19. The animal meat composition of claim 17, wherein the structured plant protein product has an average shear strength of at least 1400 grams and an average shred characterization of at least 10%.

20. The animal meat composition of claim 19, wherein the structured plant protein product comprises protein fibers substantially aligned in the manner depicted in the micrographic image of FIG. 1.

21. The animal meat composition of claim 17, wherein the animal meat is from an animal selected from the group consisting of pork, beef, lamb, poultry, wild game, fish, and mixtures thereof.

22. A simulated meat composition, the simulated meat composition comprising:
(a) a structured plant protein product comprising protein fibers that are substantially aligned, the structured plant protein product comprising an extrudate of plant protein material; and
(b) a pH-lowering agent in an amount such that the simulated meat composition has a pH below approximately 6.0.

23. The simulated meat composition of claim 22, wherein the structured plant protein product has an average shear strength of at least 1400 grams and an average shred characterization of at least 10%.

24. The simulated meat composition of claim 23, wherein the structured plant protein product comprises protein fibers substantially aligned in the manner depicted in the micrographic image of FIG. 1.
